(19) United States
(12) Patent Application Publication
(10) Pub. No.: US 2024/0240125 A1
(43) Pub. Date: Jul. 18, 2024

(54) CONTAINER ASSEMBLY FOR MICROBIOREACTOR

(71) Applicant: Beckman Coulter, Inc., Brea, CA (US)

(72) Inventors: Niklas Frische, Aachen (DE); David Meyersieck, Köln (DE); Simon Sattler, Aachen (DE); Alexander Kremers, Jülich (DE)

(73) Assignee: Beckman Coulter, Inc., Brea, CA (US)

(21) Appl. No.: 18/559,310

(22) PCT Filed: May 6, 2022
(86) PCT No.: PCT/US2022/028207

§ 371 Date: Nov. 6, 2023

Related U.S. Application Data
(60) Provisional application No. 63/301,982, filed on Jan. 21, 2022.
(60) Provisional application No. 63/227,210, filed on Jul. 29, 2021.
(60) Provisional application No. 63/185,650, filed on May 7, 2021.

(51) Int. Cl. C12M 3/06; C12M 1/00; C12M 1/12; C12M 1/34; C12M 1/36

(52) U.S. Cl. CPC C12M 23/16; C12M 23/38; C12M 27/16; C12M 37/04; C12M 41/34; C12M 41/48

(57) ABSTRACT

A gassing lid assembly enables gas-tight sealing of sample containers in general, also referred to as microplates in some embodiments, with simultaneous guided access for the pipetting unit of a dispensing/pipetting robot, also referred to as a pipettor. The component enables both gas-tight sealing and guided access for the pipetting robot. The gassing lid serves a number of purposes at the same time and provides the following advantages in a non-limiting fashion: a gas tight seal, robot integration without a gassing lid, robot integration with a gassing lid, a sealing mechanism, and anaerobic transport. Reducing the volume above reservoirs of a sample container (e.g., the volume above wells of a microplate) is advantageous in that it reduces the safety risk of high concentrations of gases such as oxygen.

This application is being filed on May 6, 2022, as a PCT International Patent application and claims the benefit of and priority to U.S. Provisional patent application Ser. No. 63/185,650, filed May 7, 2021; 63/227,210, filed Jul. 29, 2021; and 63/301,982, filed Jan. 21, 2022, the entire disclosures of which are incorporated by reference herein in their entirety.

### BACKGROUND

In many areas of biology, pharmacology, and medicine, biological systems are screened for the selection of suitable biological strains, enzymes, or suitable culture media and culture conditions, among other examples. In this context, there is a need for high sample throughputs which may be achieved via parallelization of experiments.

A microplate, or microtiter plate, is a flat plate with multiple wells that are used as small test tubes, and is one example of a device that can be utilized to achieve a high number of parallel operations. As an illustrative example, each of the individual wells may be filled with a medium, inoculated to introduce cells into the medium, and incubated at a particular temperature using a shaking incubator. Process parameters, such as a pH value, concentrations of dissolved oxygen (DO), dissolved carbon dioxide, and biomass, among other parameter values, may be continuously measured for each individual well during the growth process.

Miniaturization and parallelization in the industrial production of microorganisms have gained in economic importance in recent decades. One challenge in the cultivation of microorganisms is real-time monitoring of the process parameters of the cell cultures being produced. Controlling the supply of nutrients and the pH, and monitoring the biomass growth and the DO, allows parallel optimization of cell cultures in miniaturized bioreactors to maximize the yield of active substances, vitamins, peptides or proteins.

### SUMMARY

In general terms, the present disclosure relates to a container assembly for a microbioreactor. In one configuration, the container assembly provides an improved seal between a sample container and a gassing lid that allows a pipette tip to be inserted into a well of the sample container during agitation inside the microbioreactor. Various aspects are described in this disclosure, which include, but are not limited to, the following aspects.

One aspect relates to a component which enables a gas-tight sealing of sample containers. In some examples, the component enables gas-tight sealing of microplates.

In some further examples, in addition to enabling gas-tight sealing of sample containers, the component simultaneously provides guided access for a pipette tip. The component that enables both gas-tight sealing and guided access for the pipette tip is sometimes referred to from here on out as the “gassing lid” or “lid housing” or “lid assembly”. The gassing lid serves a number of purposes at the same time and provides the following advantages in a non-limiting fashion: a gas tight seal, robotic integration, and anaerobic transport.

The gassing lid can significantly reduce the headspace volume above the wells of a sample container (e.g., the wells of a microplate). Reducing this volume is advantageous in that it reduces the safety risk of high concentrations of gases such as oxygen.

The gassing lid provides the several advantages including: reduced headspace in the sample container safely allowing higher O₂ concentrations in the sample container; guide elements that help guide insertion of a pipette tip while agitating the sample container; multiple resilient layers with slits that open when pipette tip inserted and that close when the pipette tip is removed; sealing surfaces that distribute pressure optimally around edges of the sample container and gassing lid and allowing for partitions; and a seal that allows the sample container to be transported as a single unit for anaerobic cultivation in an aerobic workspace. Also, the gassing lid allows anaerobic cultivation inside a microbioreactor because the gassing lid prevents oxygen from entering into the cultivation wells of the sample container while in the microbioreactor.

In some examples, there are at least two types of gassing lids. A first type of gassing lid is compatible with microfluidic microplates that have microfluidics in the well bottoms coupling liquid reagents disposed in a first set of wells to a second set of wells (which have cells for cultivation). These lids include a partition separating the two sets of wells. Although the disclosure describes this first type of gassing lid as having a single partition that separates the wells into two sets, any suitable number of sets of wells separated by any suitable number of partitions are contemplated by this disclosure.

A second type of gassing lid is compatible non-microfluidic microplates (or standard microplates). These microplates do not have microfluidics for the wells.

Both microfluidic and non-microfluidic microplates allow feeding and pH control to take place simultaneously during direct nitrogen (e.g., 100% N₂) gassing of the sample container with adjustable flowrates such as, for example, between 5-50 mL/min.

Another aspect relates to enabling anaerobic or microaerophilic cultivation, sampling, feeding, and pH control when the microplates are in an aerobic environment.

One aspect is a lid assembly comprising: a lid housing having a top exterior surface and a bottom interior surface, the lid housing configured to cover a sample container; a first resilient layer disposed in the lid housing; and a sealing surface projecting from the bottom interior surface of the lid housing toward the first resilient layer to create an air-tight seal when the sealing surface is pressed against the first resilient layer.

Another aspect is a lid assembly comprising: a lid housing having a top exterior surface and a bottom interior surface, the lid housing configured to cover a sample container; one or more guide elements extending from the top exterior surface of the lid housing, each guide element having a hollow interior portion running from a top end to a bottom end, the hollow interior portion having a larger cross-sectional area at the top end than at the bottom end, and each guide element being configured to receive and guide a pipette tip; and a first layer disposed in the lid housing, the first layer including one or more first apertures aligned with a respective guide element, each first aperture being configured to open when the pipette tip is pushed through and to close when the pipette tip is removed.

Yet another aspect is a container assembly comprising: a lid assembly comprising; a lid housing with a top exterior surface and a bottom interior surface, the lid housing configured to cover a sample container; one or more guide elements extending from the top exterior surface of the lid housing, each guide element having a hollow interior portion running from a top end to a bottom end, the hollow interior portion having a larger cross-sectional area at the top end than at the bottom end, and each guide element being configured to receive and guide a pipette tip; and a first layer disposed in the lid housing, the first layer having one or more first apertures aligned with respective guide elements, each first aperture being configured to open when the pipette tip is pushed through and to close when the pipette tip is removed; and a sample container comprising a plurality of wells.

Still a further aspect is a bioreactor system comprising: a reversibly sealable sample container assembly comprising: a lid assembly comprising: a lid housing having a top exterior surface and a bottom interior surface, the lid housing configured to cover a sample container; one or more guide elements extending from the top exterior surface of the lid housing, each guide element having a hollow interior portion running from a top end to a bottom end, the hollow interior portion having a larger cross-sectional area at the top end than at the bottom end, and each guide element being configured to receive and guide a pipette tip; a first layer disposed in the lid housing, the first layer having one or more first apertures aligned with respective guide elements, each first aperture being configured to open when the pipette tip is pushed through and to close when the pipette tip is removed; a sample container comprising a plurality of wells; a platform configured to shake the sample container assembly by moving the sample container assembly within a predetermined range of motion, wherein the predetermined range of motion is within one or more interior diameters of one or more top ends of one or more of the guide elements; and pipetting robot having one or more pipette tips configured for insertion into the sample container via the one or more guide elements while the sample container assembly is being shaken.

Another aspect is a method of sealing a sample container comprising: placing a sterile layer on top of the sample container; placing a resilient layer on top of the sterile layer; pressing a lid housing on top of the resilient layer; and releasably securing the lid housing to the sample container.

Yet another aspect is a method of cultivating anaerobic cells, the method comprising: placing a sample container within an anaerobic environment; disposing a sample comprising anaerobic cells into one or more wells of the sample container while the sample container is in the anaerobic environment; creating an air-tight seal around the wells of the sample container by placing a lid assembly over the wells of the sample container; and transporting the sealed sample container to a non-anaerobic environment for cell cultivation.

Still a further aspect is a method of inserting a pipette tip into sample container while a bioreactor system is being shaken, the method comprising: placing a guide element above the sample container of the bioreactor system; shaking the bioreactor system; actuating a pipetting robot to guide the pipette tip to a narrowest region of the guide element; and guiding the pipette tip through the narrowest region of the guide element into the sample container.

Another aspect is a lid assembly for a microplate, wherein the microplate includes one or more wells, the lid assembly being configured to provide a headspace above the wells to allow gas exchange during cell cultivation, wherein the headspace above the wells is 20 mL to 400 ml.

Yet another aspect is a method of controlling gas concentrations in a headspace above wells of a microplate, the method comprising: placing a lid assembly above the microplate, the microplate including one or more wells, the lid assembly configured to provide a headspace above the wells to allow gas exchange during cell cultivation, wherein the headspace above the reservoirs is 20 mL to 400 mL; and causing a gas to flow into the headspace.

Still a further aspect is a control system for a sample container assembly with a gassing lid, the control system comprising: sensors configured to acquire measurement parameters associated with the sample container assembly; a gas supply system providing at least one gas to the gassing lid; and a controller configured to process the acquired measurement parameters and control the gas supply based upon the processed measurement parameters.

Another aspect is a method of controlling a sample container assembly with a gassing lid, the method comprising: sensing measurement parameters associated with the sample container assembly; processing the sensed measurement parameters; and controlling a gas supply of at least one gas to the gassing lid based upon the processed measurement parameters.

Yet another aspect is a computer program product, that stores in a tangible and non-transitory manner, a computer program code, that when executed by a computer controller, causes the computer controller to: sense measurement parameters associated with a sample container assembly having a gassing lid; process the sensed measurement parameters; and control a gas supply of at least one gas to the gassing lid based upon the processed measurement parameters.

Still a further aspect is a microfluidic lid assembly for creating an air-tight seal above a sample container, the lid assembly comprising: a microfluidic structure configured to be disposed over a plurality of reservoirs of the sample container to create a seal along an outside perimeter of the sample container, wherein the microfluidic structure comprises: one or more gas inlets for receiving one or more connections to one or more fluid sources; and a plurality of first microfluidic channels configured to couple the gas inlets to each of the plurality of reservoirs; wherein the microfluidic structure separates each of the reservoirs from a plurality of guide elements and a layer with apertures disposed over the reservoirs of the sample container.

In a further embodiment, the microfluidic structure is configured to individually seal each of the plurality of reservoirs. In another further embodiment, each of the plurality of first microfluidic channels is configured transport a controlled gas concentration to an individually sealed one of the plurality of reservoirs. In yet another further embodiment, at least a first subset of the plurality of first microfluidic channels is configured to convey one or more of gaseous oxygen, nitrogen, or carbon dioxide to the reservoirs. In another further embodiment, a second subset of the plurality of first microfluidic channels is configured to convey liquid reagents to the reservoirs. In yet another further embodiment, the microfluidic structure further comprises a plurality of second microfluidic channels configured to convey a gas away from the reservoirs. In another further embodiment, the plurality of guide elements and the layer form an integral unit. In yet another further embodiment, the plurality of guide elements are disposed on a guide structure that is coupled to the layer. In another further embodiment, the microfluidic lid assembly is configured to be adhered to the sample container with an adhesive. In another further embodiment, the apertures comprise slits in the layer. In yet another further embodiment, the layer comprises a resilient polymer material.

Another aspect is a sample container assembly comprising: a sample container comprising a plurality of reservoirs; and a microfluidic structure comprising: one or more gas inlets and a plurality of microfluidic channels; and wherein a bottom surface of the microfluidic structure is adhered to a top surface of the sample container. In a further embodiment, the plurality of guide elements are disposed on a guide structure that is adhered to a top surface of the layer, and a top surface of the microfluidic structure is adhered to a bottom surface of the layer.

Yet another aspect is a bioreactor system comprising: a sample container assembly comprising: a sample container comprising a plurality of reservoirs; a microfluidic structure comprising one or more gas inlets and a plurality of microfluidic channels, wherein a bottom surface of the microfluidic structure is adhered to a top surface of the sample container; one or more guide elements positioned above the microfluidic structure; a shaking table configured to shake the sample container assembly by moving the sample container assembly within a predetermined range of motion, wherein the predetermined range of motion is within one or more interior diameters of one or more top ends of one or more of the guide elements; and an automated pipettor comprising one or more pipettors configured to insert one or more pipette tips into the sample container via the one or more guide elements while the sample container assembly is being shaken.

In a further embodiment, the bioreactor system includes an upper chamber disposed above the shaking table, and a cover inlay configured to direct tempered air in the upper chamber to uniformly temper each of the plurality of reservoirs. In another further embodiment, the cover inlay includes vent holes that align with the plurality of reservoirs, the vent holes configured to direct the tempered air. In yet another further embodiment, the bioreactor system includes a lower chamber disposed below the shaking table, and one or more fans configured to circulate tempered air around the lower chamber. In another embodiment, the bioreactor system includes an upper chamber disposed above the shaking table, a lower chamber disposed below the shaking table, one or more first temperature control modules configured to temper air of the upper chamber at a first target temperature; and one or more second temperature control modules configured to temper air of the lower chamber at a second target temperature. In a further embodiment, the first temperature is set higher than the second temperature to prevent condensation in the bioreactor system.

Still a further aspect is a method of assembling a sample container assembly comprising: attaching a microfluidic structure to a top surface of a sample container; attaching a resilient layer to a top surface of the microfluidic structure; and attaching at least one guide element to a top surface of the resilient layer. In a further embodiment, the method includes adhering the microfluidic structure to the top surface of the sample container.

Another aspect is a method of inserting a pipette tip into sample container while a bioreactor system is being shaken, the method comprising: placing a guide element above a microfluidic lid assembly attached to the sample container of the bioreactor system; shaking the bioreactor system; actuating a robot arm to guide the pipette tip to a narrowest region of the guide element; and guiding the pipette tip through the narrowest region of the guide element into the sample container.

Yet another aspect is a method of cultivating anaerobic cells, the method comprising: placing a sample container with a microfluidic structure attached to a top surface of the sample container within an anaerobic environment; disposing a sample comprising anaerobic cells into one or more reservoirs of the sample container while the sample container is in the anaerobic environment; creating an air-tight seal around the reservoirs of the sample container by placing a lid assembly over the reservoirs of the sample container; and transporting the sealed sample container to a non-anaerobic environment for cell cultivation.

Still a further aspect is a method of controlling gas concentrations in a headspace above reservoirs of a microtiter plate, the method comprising: placing a microfluidic lid assembly above the microtiter plate, the microtiter plate including one or more reservoirs, the microfluidic lid assembly configured to provide a headspace above the reservoirs to allow gas exchange during cell cultivation, wherein the headspace above the reservoirs is 20 mL to 400 mL; and causing a gas to flow into the headspace.

Another aspect is a method of cultivating anaerobic cells, the method comprising: placing a sample container with a microfluidic lid assembly within an anaerobic environment; disposing a sample comprising anaerobic cells into one or more reservoirs of the sample container while the sample container is in the anaerobic environment; creating an air-tight seal around the reservoirs of the sample container by placing a lid assembly over the reservoirs of the sample container; and transporting the sealed sample container to a non-anaerobic environment for cell cultivation.

Yet another aspect is a control system for a sample container assembly with a gassing lid, comprising: sensors configured to acquire measurement parameters associated with the sample container assembly; a gas supply system providing at least one gas to the gassing lid; and a controller configured to process the acquired measurement parameters and control the gas supply based upon the processed measurement parameters.

Still a further aspect is a method of controlling a sample container assembly with a gassing lid, comprising: sensing measurement parameters associated with the sample container assembly; processing the sensed measurement parameters; and controlling a gas supply of at least one gas to the gassing lid based upon the processed measurement parameters.

Another aspect is a computer program product, that stores in a tangible and non-transitory manner, a computer program code, that when executed by a computer controller, causes the computer controller to: sense measurement parameters associated with a sample container assembly having a gassing lid; process the sensed measurement parameters; and control a gas supply of at least one gas to the gassing lid based upon the processed measurement parameters.

Yet another aspect is an automatic cell culture system, comprising: a titer module; and a bioreactor module including cell health and cell media measurement capabilities integrated with the titer module.

### BRIEF DESCRIPTION OF THE DRAWINGS

The following drawing figures, which form a part of this application, are illustrative of the described technology and are not meant to limit the scope of the disclosure in any manner.

FIG. 1 is an isometric view of a microbioreactor.

FIG. 2 is a top isometric view of a container assembly that fits inside a cultivation chamber of the microbioreactor of FIG. 1.

FIG. 3 is a bottom isometric view of the container assembly.

FIG. 4 is an exploded isometric view of the container assembly.

FIG. 5 is an exploded front elevation view of the container assembly.

FIG. 6 is a cross-sectional view of the container assembly.

FIG. 7 is a detailed view of a resilient layer of the container assembly.

FIG. 8 is a top view of an example of a sample container that includes a plurality of wells, the sample container being a component of the container assembly of FIG. 2.

FIG. 9 is a bottom view of a first example of a lid housing of the container assembly.

FIG. 10 is a bottom isometric view of the lid housing shown in FIG. 9.

FIG. 11 is a bottom isometric view of the lid housing and a sample container.

FIG. 12 is a bottom view of another example of the lid housing.

FIG. 13 is a cross-sectional view of the container assembly of FIG. 2 showing a pipette tip inserted through a guide element of the lid housing.

FIG. 14 is a cross-sectional view of the container assembly of FIG. 2 after the pipette tip has been removed from the container assembly.

FIG. 15 is a cross-sectional view of a sealing mechanism of the container assembly, the sealing mechanism being shown in an opened position.

FIG. 16 is another cross-sectional view of the sealing mechanism of FIG. 15, the sealing mechanism being shown in a closed position.

FIG. 17 is a cross-sectional view showing a seal between the lid housing and sample container of the container assembly of FIG. 2.

FIG. 18 is a cross-sectional view showing a release pin of the lid housing.

FIG. 19 is a top isometric view showing an example of the container assembly positioned on top of a base of the microbioreactor of FIG. 1.

FIG. 20 is a cross-sectional view of the container assembly of FIG. 19 on the base.

FIG. 21 is a top isometric view of the base FIG. 19.

FIG. 22 is a top isometric view showing another example of the container assembly positioned on top of a base of the microbioreactor of FIG. 1.

FIG. 23 is a cross-sectional view of the container assembly of FIG. 22 on the base.

FIG. 24 is a top isometric view of the base of FIG. 22.

FIG. 25 schematically shows an example of a computer control system of the microbioreactor of FIG. 1.

FIG. 26 is an isometric view of a mechanical system of the microbioreactor of FIG. 1 for positioning an optical sensor under the container assembly of FIG. 2.

FIG. 27 is an isometric view of a light-emitting diode array module (LAM) that can be used to illuminate the cultivation chamber of the microbioreactor of FIG. 1.

FIG. 28 is a bottom isometric view of the LAM mounted underneath the microbioreactor of FIG. 1.

FIG. 29 is a schematic diagram of the LAM.

FIG. 30 is an isometric view of a cooling plate of the LAM.

FIG. 31 is a bottom isometric view of an example of a lid housing that is adapted to cool the sample container.

FIG. 32 is a top isometric view of the lid housing of FIG. 31.

FIG. 33 illustrates a microfluidic valve configuration.

FIG. 34 illustrates an example of a method of anaerobic cultivation that can be performed using the container assembly.

FIG. 35 illustrates another example of a method of anaerobic cultivation that can be performed using the container assembly.

FIG. 36 is a graph showing dissolved oxygen, biomass gain, and added feed solution over cultivation time during a cultivation process inside the container assembly.

FIG. 37 is a graph showing pH and added NaOH volume over cultivation time during a cultivation process inside the container assembly.

FIG. 38 is a graph showing biomass over cultivation time during a cultivation process inside the container assembly.

FIG. 39 is a graph showing oxygen concentration, pH signal, and added NaOH volume over cultivation time during a cultivation process inside the container assembly.

FIG. 40 is a graph showing biomass and added feed volume over cultivation time during a cultivation process inside the container assembly

FIG. 41 is a graph showing pH, oxygen concentration, and added volume of NaOH over cultivation time during a cultivation process inside the container assembly.

FIG. 42 is a cross-sectional view of an example bioreactor system.

FIG. 43 is a perspective exploded view of the sample container assembly.

FIG. 44 is a cross-sectional view of the microfluidic structure.

FIG. 45 is a perspective view of the valve array.

FIG. 46 is a side view of the bioreactor system.

FIG. 47 is a perspective view of the bioreactor system.

FIG. 48A is a perspective view of components related to an upper chamber of the bioreactor system.

FIG. 48B is another perspective view of components related to the upper chamber of the bioreactor system.

FIG. 48C is a top view of components related to the upper chamber of the bioreactor system.

FIG. 49A is a bottom view of components related to a lower chamber of the bioreactor system.

FIG. 49B is another bottom view of components related to a lower chamber of the bioreactor system.

FIG. 50 is a block diagram of an automatic cell culture platform.

FIG. 51 illustrates an example of a method of assembling the sample container assembly.

FIG. 52 illustrates an example of a method of inserting a pipette tip into sample container while a bioreactor system is being shaken.

FIG. 53 illustrates an example of a method of cultivating anaerobic cells.

FIG. 54 illustrates another example of a method of cultivating anaerobic cells.

FIG. 55 illustrates an example of a method of controlling gas concentrations in a headspace above reservoirs of a microtiter plate.

FIG. 56 illustrates an example of a method of controlling a sample container assembly with a gassing lid

### DETAILED DESCRIPTION

Throughout all the figures, same or corresponding elements may generally be indicated by same reference numerals. These depicted embodiments are to be understood as illustrative of the invention and not as limiting in any way. It should also be understood that the figures are not necessarily to scale and that the embodiments may be illustrated by graphic symbols, phantom lines, diagrammatic representations and fragmentary views. In certain instances, details which are not necessary for an understanding of the present invention or which render other details difficult to perceive may have been omitted.

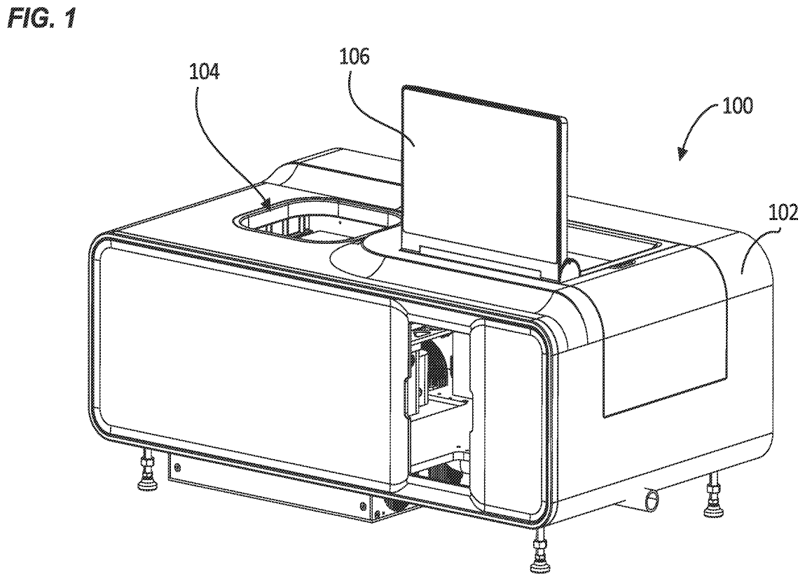

FIG. 1 is an isometric view of an example of a microbioreactor 100. As shown in FIG. 1, the microbioreactor 100 includes a housing 102 that defines a cultivation chamber 104. The microbioreactor 100 measures parameters such as biomass, pH, dissolved oxygen (DO), and fluorescence online while running a cultivation inside the cultivation chamber 104. Additionally, the microbioreactor 100 includes a touchscreen display 106 that allows a user to control the shaking speed, temperature, gas concentration, gas flow rate, and humidity inside the cultivation chamber 104. Alternatively or additionally, the microbioreactor 100 may be communicatively coupled to a separate computing device that may allow for such control.

In some aspects, the microbioreactor 100 can share similar components, features, and functionalities with the microreactors described in U.S. Pat. No. 8,268,632, titled Method and Device for Recording Process Parameters of Reaction Fluids in Several Agitated Microreactors, issued on Sep. 18, 2012, U.S. Pat. No. 8,828,337, titled Microreactor, issued on Sep. 9, 2014, U.S. Pat. No. 8,932,544, titled Microreactor Array, Device Comprising a Microreactor Array, and Method for Using a Microreactor Array, issued on Jan. 13, 2015, and U.S. Pat. No. 10,421,071, titled Microreactor System, issued on Sep. 24, 2019, the entireties of which are hereby incorporated by reference.

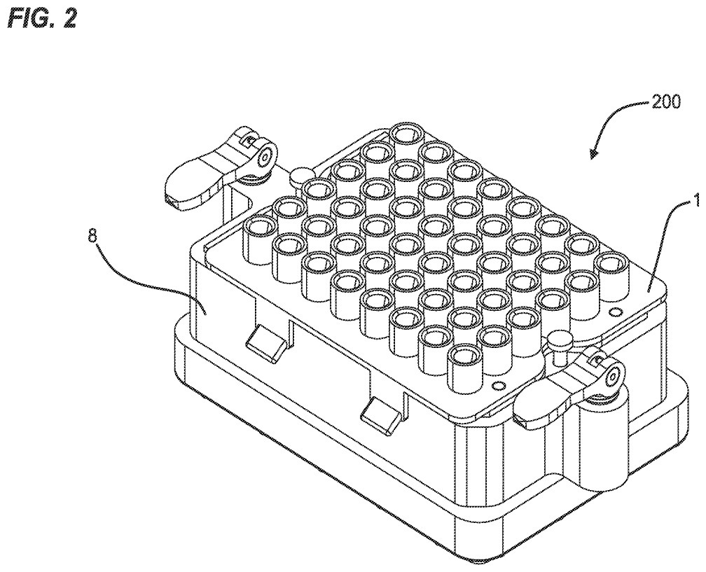

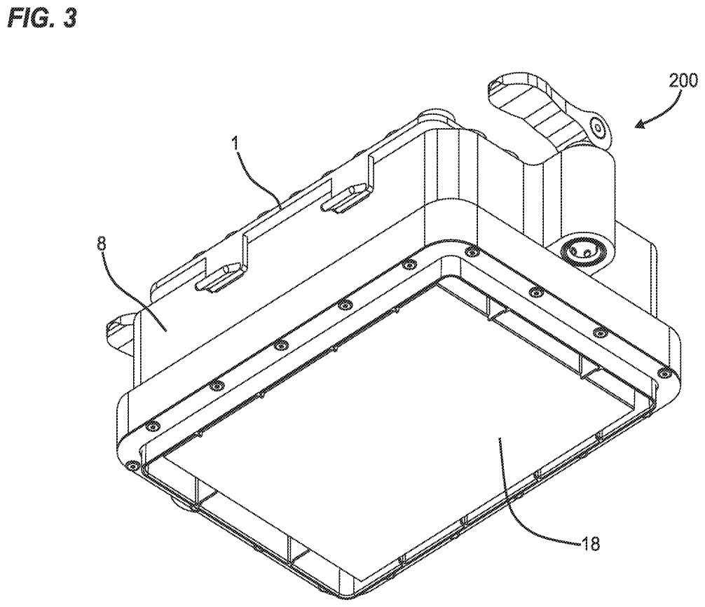

FIGS. 2 and 3 are isometric views of a container assembly 200 that fits inside the cultivation chamber 104 of the microbioreactor 100. The container assembly 200 includes a lid housing 8 that can attach or otherwise be coupled to a sample container 18. In some examples, the sample container 18 is a microplate or microtiter plate. The lid housing 8 seals the sample container 18 in a gastight manner. The lid housing 8 allows safety-critical gases to be fed into and discharged from the sample container 18 in any concentration and at any flow rate.

The container assembly 200 provides advantages that include at least a gastight seal of the sample container 18. The gastight seal of the sample container 18 enables the controlled introduction and discharge of safety-critical gases without the gases coming into contact with the atmosphere of the cultivation chamber 104 and other components of the microbioreactor 100. This enables a high level of control over gas concentrations in a headspace above the sample container 18. The headspace is the space between the sample container 18 and a bottom interior surface 28 of the lid housing 8. Furthermore, this enables maintaining high concentrations of oxygen or other gases that are unsafe (e.g., gases that are combustible, toxic, able to asphyxiate, etc.) within the container assembly 200 during cultivation and reducing safety risks such as fire or explosion. For example, the container assembly 200 allows for maintaining up to 100% pure oxygen in the headspace under reduced safety risks. Additionally, the container assembly 200 can further significantly reduce the headspace above the sample container 18 as compared to conventional systems. This further contributes to reducing the safety risks posed by, for example, high concentrations of combustible gases like oxygen by reducing the overall volume of such gases. Also, the design and selection of materials for the cultivation chamber 104 are no longer constrained by having to account for direct contact with critical gases, which reduces the technical effort required to build the microbioreactor 100.

Since the gases are fed into and discharged from the sample container 18 in a controlled manner, the flow of gases with asphyxiation potential, such as N₂ and CO₂, can be increased as needed. Additionally, the lid housing 8 reduces energy and gas consumption because only a headspace above the sample container 18 has to be humidified and gassed, rather than the entirety of the cultivation chamber 104 of the microbioreactor 100.

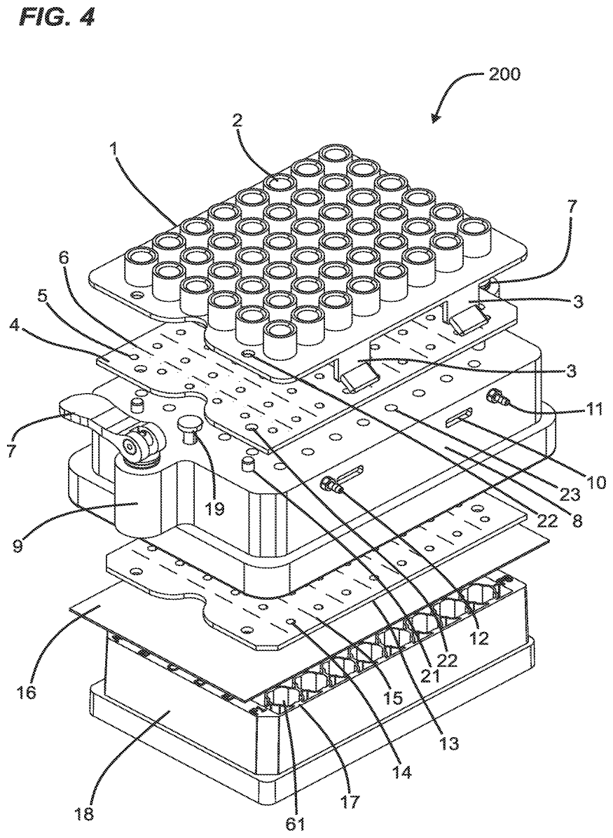

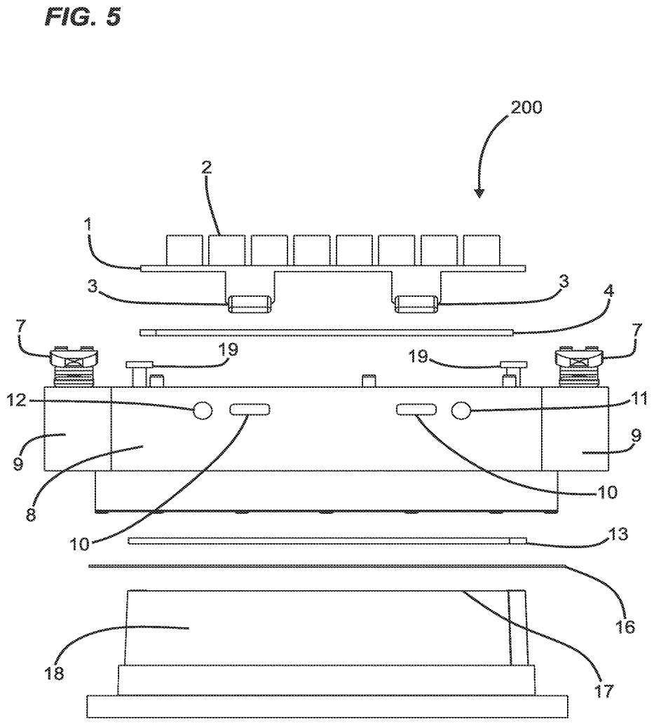

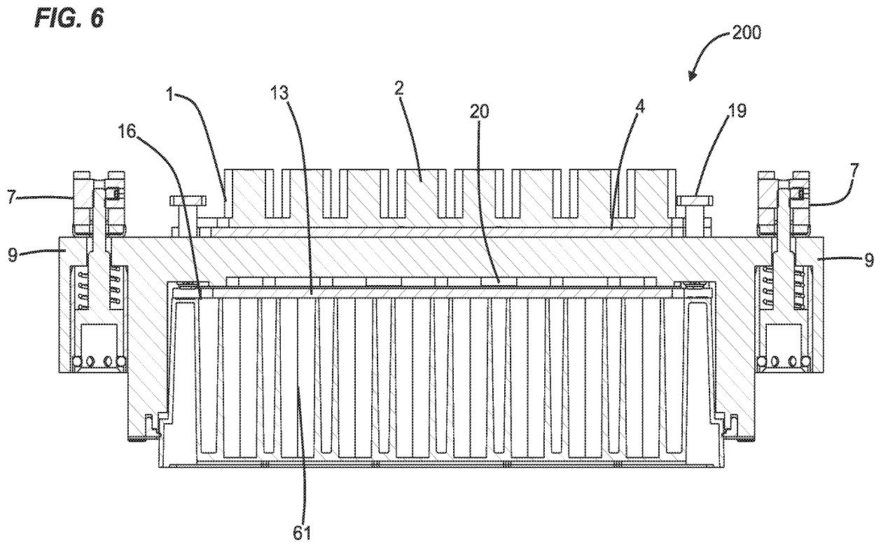

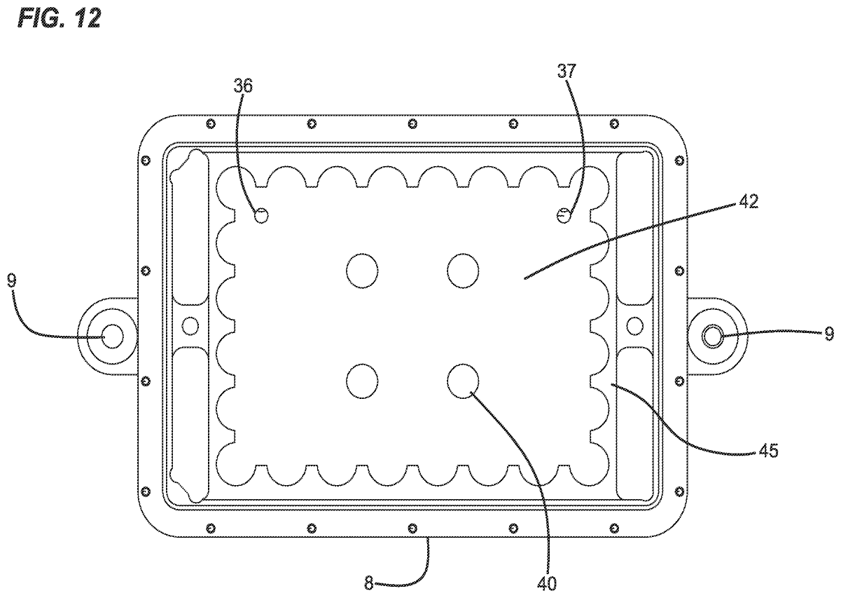

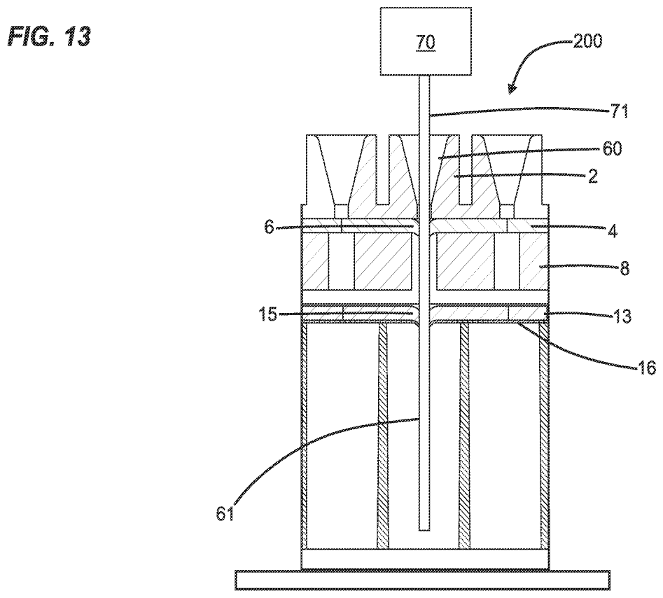

FIG. 4 is an exploded isometric view of the container assembly 200. FIG. 5 is an exploded front elevation view of the container assembly 200. FIG. 6 is a cross-sectional view of the container assembly 200. Referring now to FIGS. 2-6, the sample container 18 includes rows of wells 61 that are each configured to separately contain a cell culture or reagent. The sample container 18 is completely covered by the lid housing 8, but is still accessible for one or more pipette tips (see pipette tip 71 shown in FIG. 13) to feed liquids into the wells 61 or to take probes from the wells 61. Thus, the lid housing 8 has an assembly which allows the pipette tip 71 to enter the container assembly 200 without allowing the gas atmosphere to escape from the container assembly 200. The lid housing 8 has through-holes 23 above each of the wells 61 for the pipette tip 71 to access each well.

A guide structure 1 is releasably and reversibly coupled to the lid housing 8. The guide structure 1 includes a plurality of guide elements 2. The guide structure 1 further includes one or more attachment mechanisms 3 that are structured to engage corresponding slots 10 on the lid housing 8 to removably attach the guide structure 1 onto the lid housing 8.

The attachment mechanisms 3 are structured to flex against an exterior surface of the lid housing 8, and snap fit into the corresponding slots 10. The attachment mechanisms 3 can include handles that allow a user to disengage the attachment mechanisms 3 from the corresponding slots 10, and to thereby release the guide structure 1 from the lid housing 8. In alternative examples, the guide elements 2 form an integral part of the lid housing 8.

A first resilient layer 13 is positioned between the lid housing 8 and a sterile layer 16. A second resilient layer 4 is positioned between the lid housing 8 and the guide structure 1.

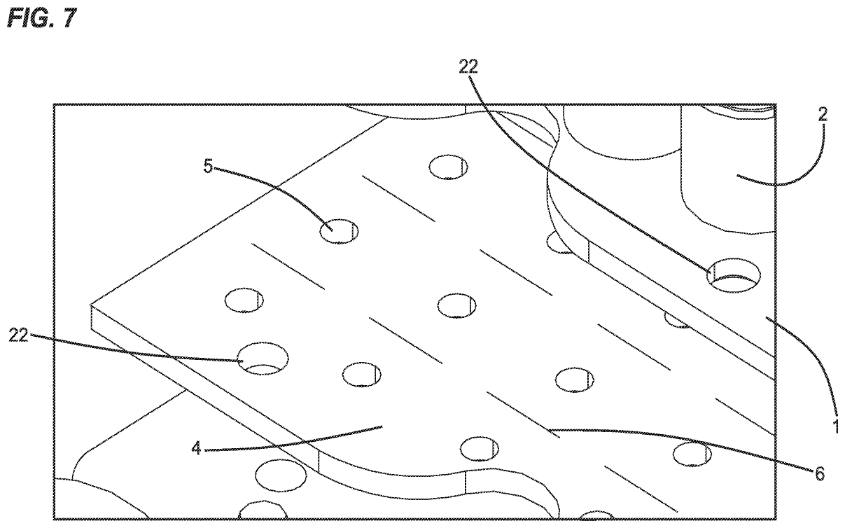

FIG. 7 is a detailed view of the second resilient layer 4. While the figures show the first and second resilient layers 13, 4 as being identical, in some examples they are not. Referring now to FIGS. 4 and 7, the first and second resilient layers 13, 4 each include apertures 15, 6. While the apertures 15, 6 are illustrated as slits having a linear shape, alternative shapes and configurations for the apertures 15,6 are possible.

As shown in FIG. 4, the apertures 6 of the second resilient layer 4 align with the through-holes 23, and the apertures 15 of the first resilient layer 13 align with the through-holes 23. The components of the container assembly 200 are arranged such that a pipette tip 71 can be inserted through a guide element 2, through an aperture 6 of the second resilient layer 4, through a through-hole 23 of the lid housing 8, and through an aperture 15 of the first resilient layer 13, such that the pipette tip 71 can pierce the sterile layer 16, and reach a well 61 of the sample container 18. In some examples, the guide element 2, the second resilient layer 4, the lid housing 8, and the first resilient layer 13 form a gassing lid for the container assembly 200.

At least the second resilient layer 4 includes holes 22 that align with pins 21 on a top exterior surface of the lid housing 8. Cooperation between the holes 22 on the second resilient layer 4 and the pins 21 allows the second resilient layer 4 to be fixed relative to the lid housing 8.

In some examples, the guide structure 1 includes holes 22 that align with the pins 21 on the top exterior surface of the lid housing 8. Cooperation between the holes 22 on the guide structure 1 and the pins 21 allows the guide structure 1 to be fixed relative to the lid housing 8.

At least the first resilient layer 13 includes holes 14 that allow for gases to pass through the first resilient layer 13. The holes 14 are positioned adjacent to the apertures 15. In the example depicted in the figures, the second resilient layer 4 also includes holes 5 that are adjacent to the apertures 6. For clarity, the holes 5 do not serve a purpose. The holes 5 are not aligned with the holes 14 of the first resilient layer 13, the through-holes 23 of the lid housing 8, or the guide elements 2 of the guide structure 1, and the holes 5 do not allow for gases to escape from the container assembly 200. Accordingly, the container assembly 200 is airtight. The holes 5 exist in the second resilient layer 4 only so that the same part can be manufactured for use as both the first and second resilient layers 13, 4. Accordingly, the holes 5 are optional. Thus, in alternative examples, the second resilient layer 4 does not include the holes 5.

The first and second resilient layers 13, 4 are made from a resilient material such as silicone. The resilient material of the first and second resilient layers 13, 4 can help reduce contamination and evaporation inside the container assembly 200, and maintain gas concentrations at desired levels by not allowing mixing within the container assembly 200.

The first and second resilient layers 13, 4 are resilient in that they are capable of recovering their size and shape after deformation. For example, the apertures 15, 6 are self-healing apertures that are configured to open when the pipette tip 71 (see FIG. 13) is inserted therethrough, and the apertures 15, 6 are configured to self-heal and close when the pipette tip 71 is removed therefrom, as will be discussed in more detail below.

It can be advantageous to use at least two resilient layers (i.e., the first and second resilient layers 13, 4) in the configuration described herein. For example, a bottom resilient layer (e.g., the first resilient layer 13) can keep the wells 61 in the sample container 18 covered for sterility and prevent evaporation. A top resilient layer (e.g., the second resilient layer 4) can provide additional sterility, and seals the top of the lid housing 8 (covering the through-holes 23 at all times when not pierced by a pipette tip) so as to help regulate the gas concentration in a headspace 20 (see FIG. 6) of the lid housing 8. In some examples, any number of additional layers may be used for added benefits (e.g., increased sterility and/or sealing).

The sterile layer 16 is made from a sterile material such as a cellulose membrane, or any other suitable layer that is biocompatible and capable of maintaining sterility. For example, the sterile layer 16 can be made from a fabric having a pore size that is small enough to not be permeable to microorganisms and water vapor, and that is large enough to be permeable to gases.

An adhesive can be used to secure the sterile layer 16 around a perimeter of the sample container 18. As an illustrative example, the adhesive can be applied to either the sterile layer 16 or the sample container 18 using an applicator or similar means. In some examples, the entire sterile layer 16 is an adhesive that can be directly applied onto the sample container 18.

The sterile layer 16 provides a sterile boundary between the sample container 18 and the cultivation chamber 104 of the microbioreactor 100. Advantageously, the cultivation chamber 104 does not have to be kept sterile at all times to prevent contamination of the cell cultures inside the wells 61 of the sample container 18. Additionally, the sterile layer 16 can reduce evaporation while also being permeable to gases such as O₂, N₂, CO₂, air, and the like.

The sterile layer 16 is useful in preventing or at least reducing contamination of cell cultures within the wells 61 of the sample container 18 (especially prior to first piercing by a pipette tip as described below), and is also useful in preventing or at least reducing evaporation from leaving the wells 61 of the sample container 18. As will be described further below, when taking a sample or adding suspending agents to the wells 61 of the sample container 18, the pipette tip 71 pierces the sterile layer 16 of the container assembly 200.

Although the holes that result from the pipette tip 71 piercing the sterile layer 16 may reduce some of the effects provided by the sterile layer 16 mentioned above, the reduction in effectiveness is mitigated in that the holes created from the pipette tip 71 piercing the sterile layer 16 are relatively small. Additionally, one or more resilient layers (e.g., the first and second resilient layers 13, 4 above the sterile layer 16) can help seal the headspace 20 above the wells 61 (e.g., sample reservoirs) in the sample container 18. The one or more resilient layers can also help reduce contamination after the sterile layer 16 is pierced, and further reduce evaporation.

Additionally, the one or more resilient layers provide an air-tight seal that allows for controlling and maintaining necessary gas concentrations in the headspace 20 above the wells 61 in the sample container 18. The lid housing 8 is configured to provide the headspace 20 above the wells 61 to allow gas exchange during cell cultivation. In some examples, the headspace 20 provided by the lid housing 8 can range from 20 mL to 400 mL. In some further examples, the headspace 20 provided by the lid housing 8 can range from 60 mL to 90 mL for a first type of gassing lid that has a partition and is configured to work with plates having microfluidics, as will be described further. In some further examples, the headspace 20 provided by the lid housing 8 can range from 80 to 120 mL for a second type of gassing lid that is configured to work with plates having no microfluidics and is therefore not partitioned, as will be described further.

As shown in FIGS. 4 and 5, the lid housing 8 can include gas ports 11, 12 that allow gas to enter and exit the headspace 20 above the wells 61 in the sample container 18. For example, the gas port 11 may be an inlet port and the gas port 12 may be an outlet port (or vice versa). In some examples, the gas inlet port may be coupled to a device that mixes two or more gases (e.g., oxygen, carbon dioxide, and nitrogen) to achieve a gas mixture having desired concentrations for supplying to the gas mixture to the headspace 20. The gas outlet port is used to exhaust gas from the headspace 20 at a desired flow rate (e.g., matching a flow rate of the gas inlet once a desired pressure has been achieved). Although not illustrated, in some examples, the lid housing can include multiple inlets for different gases. For example, it may include separate inlets for oxygen, carbon dioxide, and nitrogen.

Although this disclosure references gas inlets/ports and gas sources for simplicity, it contemplates that the gas sources may be in liquid or partially liquid form, and that the fluid flowing into the gas inlets and through the corresponding channels/pipes may be in liquid or partially liquid form.

The examples depicted in FIGS. 4 and 5 show the lid housing 8 as including two gas ports: a gas inlet port and a gas outlet port. In such examples, the lid assembly 8 can be used for non-microfluidic applications. In alternative examples, such as when the lid housing 8 is adapted for microfluidic applications, the lid housing 8 can include an additional gas port for introducing or removing a pressurizing gas to/from the space above reservoir wells (such space being partitioned from the headspace above cultivation wells) so as to control fluid flow of reagents from the reservoir wells to the cultivation wells, as will be described further below. Although this disclosure discloses a certain number of gas ports, any suitable number of gas ports is contemplated. For example, additional gas ports may be included for different gases such as an inlet port for oxygen, an inlet port for CO₂, an inlet port for nitrogen, and the like.

In some examples, the gas ports allow the headspace 20 above the wells 61 in the sample container 18 to have an oxygen concentration that ranges from 0% to 100% such that the container assembly 200 can be used for cultivating an entire range of cells from extreme anaerobes to aerobic organisms by allowing for a wide range of oxygen concentrations in the headspace 20. In some examples, the gas ports can be used to adjust the oxygen concentration in the headspace 20 to a level between 0% and 5%, 0% and 10%, or 0% and 20%.

The lid housing 8 also includes eccentric levers 7 and sealing mechanisms 9 to secure and seal the lid housing 8 to the sample container 18. The eccentric levers 7 and sealing mechanisms 9 will be discussed in more detail below.

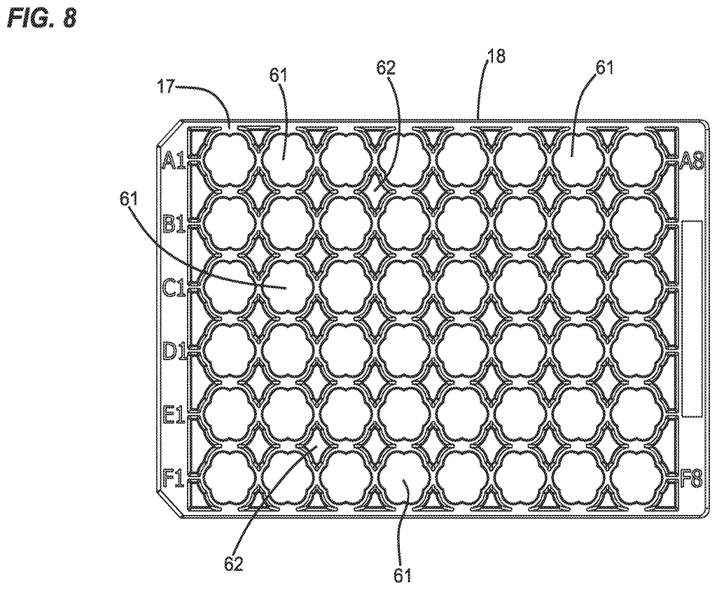

FIG. 8 is a top view of the sample container 18. As shown in FIG. 8, the sample container 18 includes a plurality of wells 61 that are arranged in rows. In the example shown in FIG. 8, the sample container 18 includes a total of 48 wells allowing a user to perform 48 parallel cultivations. Alternative sizes for the sample container 18 are possible. For example, the sample container 18 may be sized to include any of 6, 12, 24, 96, 384, or 1536 wells, and any number of wells therebetween, or any suitable number of wells.

As further shown in FIG. 8, the sample container 18 includes a sealing surface 17 that surrounds the wells 61. The sealing surface 17 includes a plurality of curved edges that are linked together, and that are semi-circularly shaped around the perimeters of the wells 61.

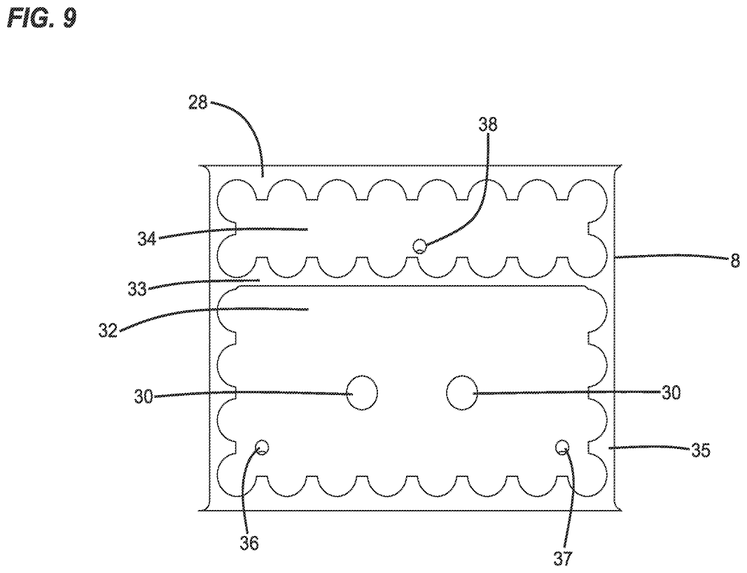

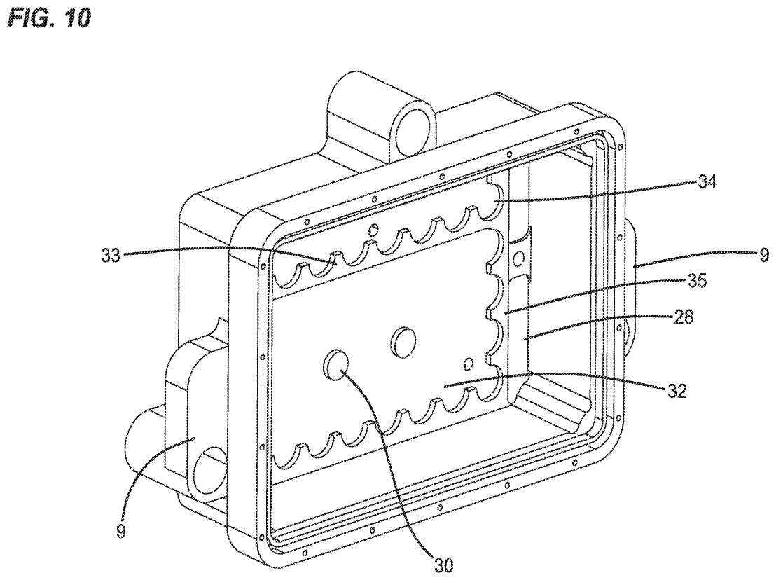

FIG. 9 is a bottom view of a first example of a lid housing 8 that is partitioned for a microplate with microfluidics. FIG. 10 is a bottom isometric view of the example of the lid housing 8 that is partitioned for a microplate with microfluidics. FIG. 11 is a bottom isometric view of the lid housing 8 that is partitioned for a microplate with microfluidics relative to the sample container 18. As shown in FIGS. 9-11, a sealing surface 35 projects from a bottom interior surface 28 of the lid housing 8. The sealing surface 35 is configured to contact and push down on the first resilient layer 13 (see also FIG. 6), which is thus caused to be compressed against the sealing surface 17 of the sample container 18, thereby forming an airtight seal between an inside perimeter of the lid housing 8 and the sample container 18. This illustrates yet another advantage of the first resilient layer 13.

The sealing surface 35 is elevated relative to first and second recessed areas 32, 34. The sealing surface 35 can act as a boundary structure between the first and second recessed areas 32, 34 and an exterior of the container assembly 200.

The sealing surface 35 conforms to the shape of the sealing surface 17 of the sample container 18 to apply an even pressure along the edges of the sealing surface 17.

The sealing surface 35 is configured to surround the edges of the wells 61 with minimal intrusion inward.

A threshold amount of pressure is needed between the lid housing 8 and sample container 18 to create an air-tight seal between the sealing surfaces 17, 35. Increasing the surface area of contact between the sealing surfaces 17, 35 increases the threshold amount of pressure needed to create the air-tight seal, which can compromise the structural integrity of the sample container 18. The shape of the sealing surfaces 17, 35 reduces the contact area to an optimal region surrounding the wells 61 that minimizes the threshold amount of pressure needed to form an air-tight seal between the lid housing 8 and sample container 18.

In some examples, the sample container 18 is a microfluidic sample container. In such examples, the rows A and B of the sample container 18 (see FIG. 8) can serve as reservoir wells (e.g., wells containing media, reagents, nutrients, pH regulation liquids, or any other suitable liquids) that can feed into the other wells having cells for cultivation that are fed via microfluidic pumping processes. While an atmosphere with specific properties is to be produced in the wells 61 that are used for cultivation (referenced herein as “cultivation wells”), pressure may need to be applied to the reservoir wells so that the pumping process can be carried out. That is, a pressuring gas (e.g., nitrogen) may be introduced into the space above the reservoir wells to increase pressure and thereby cause fluid from the reservoir wells to be conveyed into the cultivation wells via microfluidic channels, as will be described further below. In order to maintain desired pressures and gas concentrations in the headspace above the cultivation wells, these regions of the sample container 18 have to be separated. Thus, a partition 33 is used to separate the first and second recessed areas 32, 34, as shown in FIGS. 9-11.

As shown in FIGS. 9-11, the partition 33 is on the bottom interior surface 28 of the lid housing 8 to create separate sections of the wells 61 that are sealed off from each other. In some examples, the sealing surface 35 and the partition 33 are continuous with one another.

As an illustrative example, the partition 33 can define the first recessed area 32 which is designated for the cultivation wells (for clarity, the first recessed area 32 is what defines the headspace 20 above the cultivation wells), and further defines the second recessed area 34 which is designated for reservoir wells.

While two separate recessed areas are shown in this example, the lid housing 8 may include additional partitions to further subdivide the wells 61. For example, a first set of wells for cultivations that need a first concentration of a gas such as oxygen may be subdivided by another partition from a second set of wells for cultivations that need a second concentration of the gas (e.g., oxygen) that is different from the first concentration.

The sealing surface 35 and partition 33 are made from a rigid material that can be pressed against the first resilient layer 13 to compress the first resilient layer 13 and thereby provide the desired level of sealing. In some examples, the sealing surface 35 and partition 33 are made from a rigid polymer material such as polyether ether ketone (PEEK).

As shown in the example provided in FIG. 9, the lid housing 8 includes two openings 36, 37 that are each respectively connected to a gas port 11, 12 shown in FIGS. 4 and 5. One of these openings is an inlet for feeding gas, the other opening is an outlet for exhausting gas. The openings 36, 37 are provided in the first recessed area 32, and can thus be used for feeding gas with a controlled concentration of air, oxygen, nitrogen, or CO2 to the cultivation wells in the sample container 18. In some examples, this gas may be humidified to a desired level. A third opening 38 is provided in the second recessed area 34, and can be connected to a third gas port 25 on the lid housing 8 (see FIG. 23) for feeding a pressurizing gas to the reservoir wells in the sample container 18 so as to pressurize the headspace above the reservoir wells and thus cause liquid from the reservoir wells to move into the cultivation wells via microfluidic channels. In this example, the lid assembly 8 is configured for microfluidic applications.

The lid housing 8 environmentally seals the sample container 18. The mixed gases that form a desired atmosphere for the cell cultivations are guided under the lid housing 8 to pass over the wells 61. In cases where the sample container 18 includes reservoir wells for feeding cultivation wells in the sample container 18, the reservoir wells can be sealed off from the cultivation wells using the partition 33 of the lid housing 8.

The partition 33 allows for a pressure applied on top of the reservoir wells to be different from a pressure applied on top of the wells that are used to cultivate the microbial cultures. The partition 33 also allows for preventing mixing of the gases over the reservoir wells with components in the cultivation wells, and thus allows for gas in the headspace above the cultivation wells to be regulated.

Still referring to FIGS. 9-11, the lid housing 8 can include one or more posts 30 that push down on the first resilient layer 13 covering the sample container 18 so as to ensure that the first resilient layer 13 does not deform beyond a prescribed limit during cultivation. For example, the first resilient layer 13 can be made of a material that may be susceptible to expanding and thus deforming (either temporarily or permanently) in an outward direction when it is exposed to heat and gases above a certain threshold. The posts 30 project from bottom interior surface 28 in the first recessed area 32 which covers the wells 61 that are configured for cell cultivation. The posts 30 extend toward the wells 61 to help prevent the first resilient layer 13 from deforming and pushing upwards (e.g., beyond a threshold amount) due to the heat, gases, or other forces that emit from the system as a whole including the wells 61 during cultivation. In some examples, the posts 30 do not extend as far as the partition 33 or the sealing surface 35, and thus do not touch or push against the resilient layer 13 when the seal is formed. Rather, the posts 30 may only touch when the resilient layer 13 deforms more than a threshold amount.

While FIGS. 9-11 show two posts on the bottom interior surface 28 of the lid housing 8, in alternative examples the lid housing 8 may include only one post, or may include more than two posts. Thus, the arrangement of the posts 30 that is shown in FIGS. 9 and 10 is provided as an illustrative example, and the lid housing 8 is not limited to this particular arrangement.

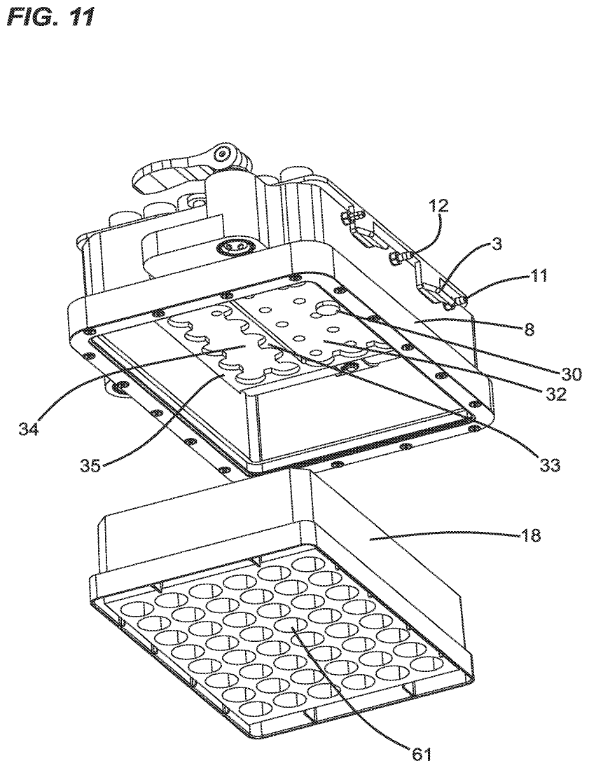

FIG. 12 is a bottom view of another example of the lid housing 8. In this example, the bottom interior surface of the lid housing 8 does not include the partition 33 shown in FIGS. 9-11, such that there is only a single recessed area 42 on the bottom interior surface of the lid housing 8. A sealing surface 45 surrounds the recessed area 42. The sealing surface 45 is substantially similar or the same as the sealing surface 35. In this example, the bottom interior surface of the lid housing 8 includes four posts 40 that project from the recessed area 42.

In the example illustrated in FIG. 12, the lid housing 8 includes two openings 36, 37 that are connected to the gas ports 11, 12 shown in FIGS. 4 and 5. The openings 36, 37 each correspond to one of an inlet gas port and an outlet gas port (e.g., gas ports 11, 12) and are provided in the single recessed area 42. The openings 36, 37 can be used for feeding gas with a controlled concentration of air, oxygen, nitrogen, or CO₂ to the cultivation wells and exhausting the gas. In this example, the lid assembly 8 is configured for non-microfluidic applications.

FIG. 13 is a cross-sectional view of the container assembly 200 showing a pipette tip 71 inserted through a guide element 2 of the lid housing 8. In this example, a pipetting robot 70 controls the movement of the pipette tip 71. FIG. 14 is a cross-sectional view of the container assembly 200 after the pipette tip 71 has been removed from the container assembly 200.

Although the pipetting robot 70 is described for controlling the movement of the pipette tip 71, the disclosure herein contemplates that sampling and introducing fluids into the wells 61 of the sample container 18 may also be performed manually. For example, a user may manually insert one or more pipette tips 71 into one or more wells 61 for sampling a fluid from the one or more wells 61 or introducing a fluid into the one or more wells 61.

As shown in FIGS. 13 and 14, the guide elements 2 each define a hollow interior portion 60 that can help guide the pipette tips 71 toward a specific location in the sample container 18 (e.g., a well 61). In some examples, the hollow interior portions 60 have a conical or frustoconical shape to help guide the pipette tip 71 through the various layers and components of the container assembly 200. This is especially advantageous when pipette tip 71 is inserted and removed during agitation or shaking of the container assembly 200 by the microbioreactor 100 when inside the cultivation chamber 104 during cultivation and/or fermentation.

During the orbital shaking motion of the container assembly 200, the pipette tip 71 may be inserted into a guide element 2 above a desired well. As soon as the diameter of the hollow interior portion 60 becomes smaller than an agitation (e.g., shaking) diameter inside the cultivation chamber 104, there is direct contact between the pipette tip 71 and the guide element 2. Due to the flexibility of the pipette tip 71 and of the connection to the pipetting robot 70, the pipette tip 71 is guided to the narrowest part of the guide element 2 and finally through the guide element 2. Thereafter, the pipette tip 71 can be pushed through the various layers and components of the container assembly 200 to reach a well 61 of the sample container 18.

For example, the pipette tip 71 can pass through an aperture 6 of the second resilient layer 4, a through-hole 23 of the lid housing 8, and an aperture 15 of the first resilient layer 13 until it reaches the sterile layer 16. The pipette tip 71 is sufficiently rigid so as to pierce a hole in the sterile layer 16, and thereafter reach a well 61 on the sample container 18. Accordingly, the guide elements 2, the through-holes 23 of the lid housing 8, and the various layers may serve to accurately position the end of the pipette tip 71 at a steady location over the sample container 18, and the size of the hole can be limited to the size of the pipette tip 71 as the sample container 18 is shaken such that the hole is not enlarged due to the shaking of the sample container 18, and accordingly, the size of the hole formed from the pipette tip 71 passing through is minimized.

Furthermore, the accurate positioning of the pipette tip 71 by the guide elements 2 ensures that the sterile layer 16 is not pierced in multiple locations over the same well during multiple insertions of the pipette tip 71. This is an advantageous feature because a single experiment may include several hundred pipetting tip insertions over a single well, and multiple holes in the sterile layer 16 over the same well may increase the risk of contamination.

The microbioreactor 100 includes an actuator system that is configured to move the container assembly 200 in an orbital fashion. Continuous shaking improves the aeration of the wells 61 and prevents sedimentation inside the wells. Thus, interrupting the shaking while pipetting into a well or out of a well is not desirable. In order to prevent the apertures 15, 6 of the first and second resilient layers 13, 4 from wearing out, the pipette tip 71 must hit the middle of the apertures 15, 6 to avoid harming the flanks of the apertures 15, 6.

To guide the pipette tip 71 to a through-hole 23 in the lid housing 8 and the middle of the apertures 15, 6 in the first and second resilient layers 13, 4, the pipette tip 71 is guided by the guide elements 2 of the guide structure 1. The guide elements 2 act like funnels for guiding the pipette tip 71 to the through-holes 23 and the middle of the apertures 15, 6 and keeping the pipette tip 71 centered in this location during agitation of the container assembly 200. After the pipette tip 71 is removed from the apertures 15, 6, the apertures close by themselves through the elastic nature of the first and second resilient layers 13, 4.

In some examples, the actuator system of the microbioreactor 100 is configured to move the container assembly 200 in an orbital fashion within a range of 600 RPM to 1000 RPM, and with an agitation diameter of 1-6 mm. In some further examples, the actuator system of the microbioreactor 100 is configured to move the container assembly 200 in an orbital fashion within a range of 600 RPM to 800 RPM, and with an agitation diameter of 1-5 mm. In some further examples, the actuator system of the microbioreactor 100 is configured to move the container assembly 200 in an orbital fashion within a range of 100 RPM to 1000 RPM, and with an agitation diameter of 1-5 mm. In some further examples, the actuator system of the microbioreactor 100 is configured to move the container assembly 200 in an orbital fashion within a range of 600 RPM to 800 RPM, and with an agitation diameter of 3 mm. In some further examples, the actuator system of the microbioreactor 100 is configured to move the container assembly 200 in an orbital fashion within a range of 100 RPM to 2000 RPM, and with an agitation diameter of 1-30 mm. In some further examples, the actuator system of the microbioreactor 100 is configured to move the container assembly 200 in an orbital fashion within a range of 0 RPM to 2000 RPM. In some further examples, the actuator system of the microbioreactor 100 is configured to move the container assembly 200 in an orbital fashion within a range of 100 RPM to 1500 RPM. In some examples, the agitation diameter may be between 1 and 6 mm.

After the sterile layer 16 is pierced by the pipette tip 71, the first resilient layer 13 maintains a seal over the wells 61 of the sample container 18. For example, the first resilient layer 13 includes an aperture 15 over each of the wells 61, and the apertures 15 are “self-healing” in that they can automatically seal in on themselves when not penetrated by the pipette tip 71.

The first and second resilient layers 13, 4 can be made from any suitable resilient and compliant material. In some examples, the first and second resilient layers 13, 4 are silicone films, which provide suitable resilience and compliance. Alternatively, the first and second resilient layers 13, 4 can be made from soft polymers, or hard polymers blended with a softener.

The pipette tip 71 is precisely guided into an aperture 15 of the first resilient layer 13 by the guide elements 2 described above. Each of the apertures 15 opens due to the pressure of the pipette tip 71, such that said pipette tip 71 can pierce the sterile layer 16 and dip into a well 61 as shown in FIG. 13. After the pipetting procedure has ended, the pipette tip 71 is pulled out of the well 61. Without the pressure exerted by the pipette tip 71, the aperture 15 of the first resilient layer 13 closes on its own in a gas-tight manner and thus provides a seal over the sterile layer 16 when damaged.

FIGS. 13 and 14 show the self-healing nature of the apertures 15 of the first and second resilient layers 13, 4. In FIG. 13, the apertures 15, 6 are opened when the pipette tip 71 is inserted, and in FIG. 14, the apertures 15, 6 are closed after the pipette tip 71 is removed.

The through-holes 23 can cause gases to leak from the headspace 20 of the lid housing 8. Thus, the second resilient layer 4 is applied over the through-holes 23 of the lid housing 8. In some examples, the second resilient layer 4 is self-adhesive on at least one side.

The apertures 6 of the second resilient layer 4 are aligned above each respective through-hole 23 of the lid housing 8. The apertures 6, like the apertures 15, are self-healing such that they are configured to open when the pipette tip 71 is pushed through, and after the pipetting procedure, the apertures 6 close on their own to seal the headspace 20.

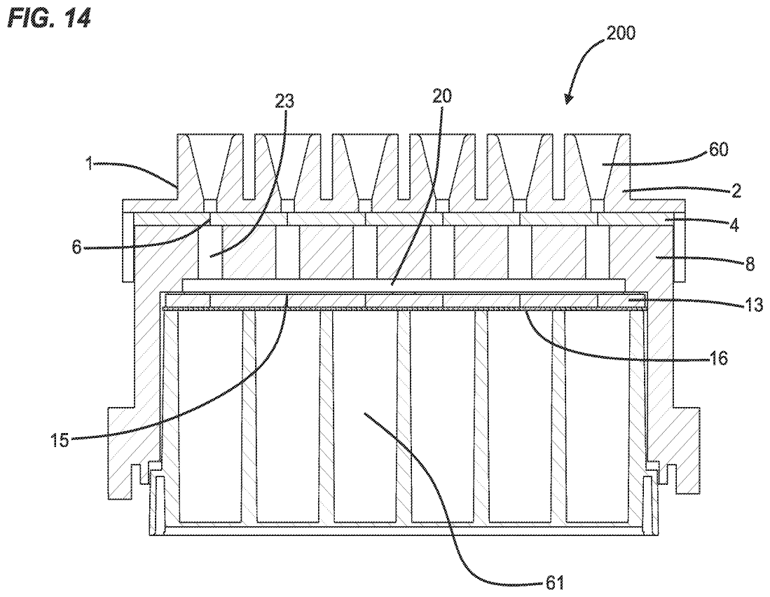

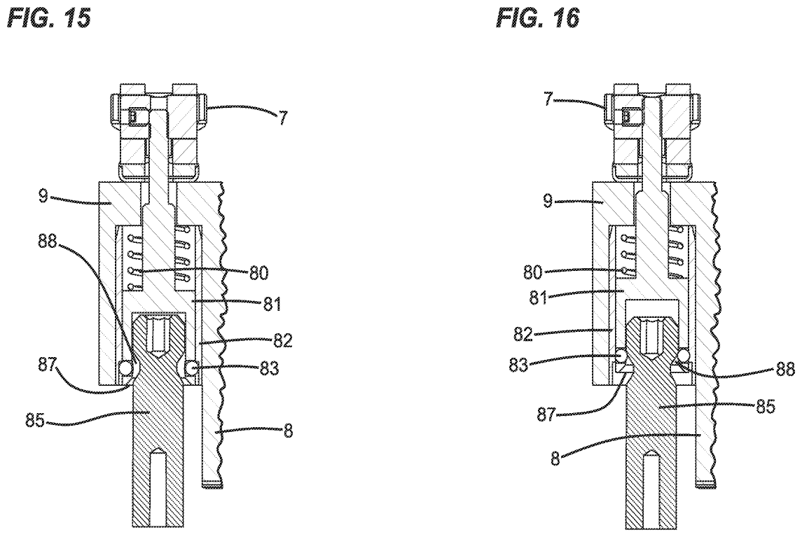

FIG. 15 is a cross-sectional view showing a sealing mechanism 9 in an opened condition. FIG. 16 is a cross-sectional view showing the sealing mechanism 9 in a closed condition. The sealing mechanism 9 is configured to press the lid housing 8 onto the sample container 18 with a sufficient amount of pressure to create an air-tight seal between the lid housing 8 and the sample container 18, without damaging to the sample container 18.

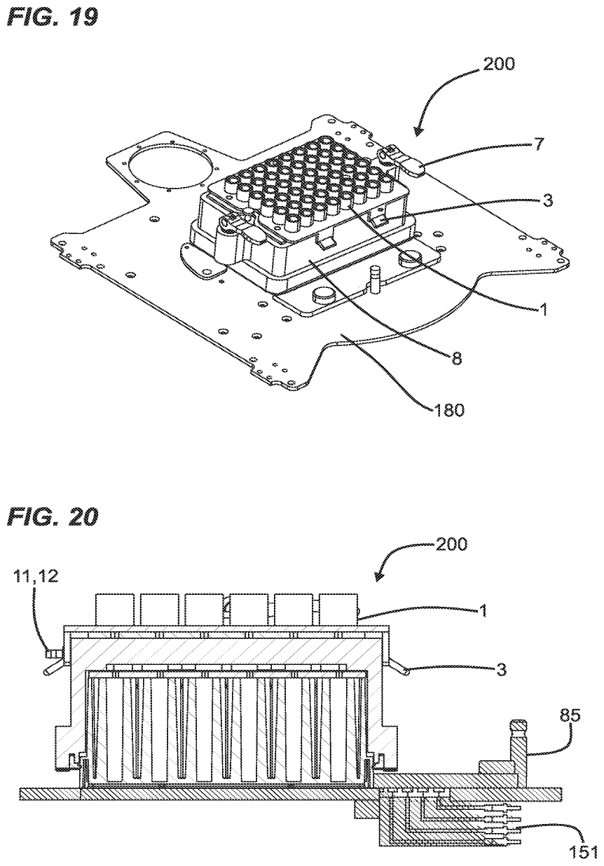

The sealing mechanism 9 includes a press sleeve 82 that is mounted inside the sealing mechanism 9 by a press-fit connection. The press sleeve 82 is vertically aligned with a ball sleeve 81 inside the sealing mechanism 9. The ball sleeve 81 terminates in a shoulder 87 that engages radially guided balls 83. Based on the vertical position of the shoulder 87, the radially guided balls 83 open or close the seal between the lid housing 8 and a base post 85 (see FIG. 19).

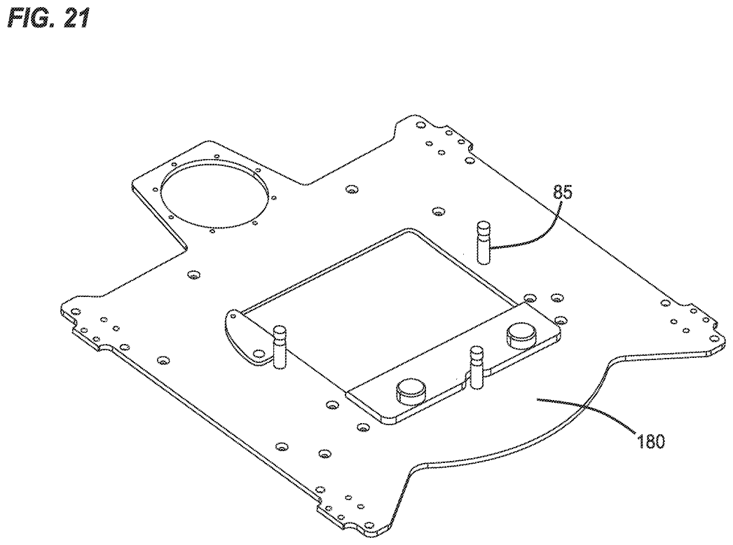

The ball sleeve 81 is biased by a spring 80 to be pushed down in the opened position. When the eccentric lever 7 is actuated, the shoulder 87 is pulled up. The diameter of the press sleeve 82 is decreased when the radially guided balls 83 are pulled upward which causes the radially guided balls 83 to be displaced in a radial direction toward a groove 88 of the base post 85 of an orbital shaking platform 180 (see FIGS. 19 and 21). This produces a form fit between the lid housing 8 and the base post 85, and thereby attaches the lid housing 8 to the base post 85.

Additionally, the sealing mechanisms 9 causes the lid housing 8 to press onto the sample container 18 to create a sealed gas atmosphere above the sample container 18 when the sample container 18 is held inside the cultivation chamber 104 of the microbioreactor 100. Due to the generated torque, the eccentric lever 7 is self-locking in the closed position.

When the eccentric lever 7 is operated to move from the closed position to the opened position, the radially guided balls 83 are pushed downwards where the diameter of the press sleeve 82 is increased, such that the radially guided balls 83 expand in a radial direction away from the groove 88 to release the lid housing 8 from the base post 85. Thus, the sealing mechanism 9 provides an easy way to attach and detach the lid housing 8 from the base post 85, as well as to press the lid housing 8 onto the sample container 18, and to release it therefrom.

When the lid housing 8 is pressed onto the sample container 18, a seal is created between the lid housing 8 and sample container 18 by the geometrically unique elevations of the sealing surface 35 and partition 33 inside the lid housing 8, and the sealing surface 17 on the sample container 18. The shape of the sealing surfaces 17, 35 reduces the pressure from the sealing mechanism 9 and eccentric levers 7 needed for pressing the lid housing 8 onto the sample container 18, allowing the lid housing 8 to seal the sample container 18 in a gas-tight manner without damaging the sample container 18.

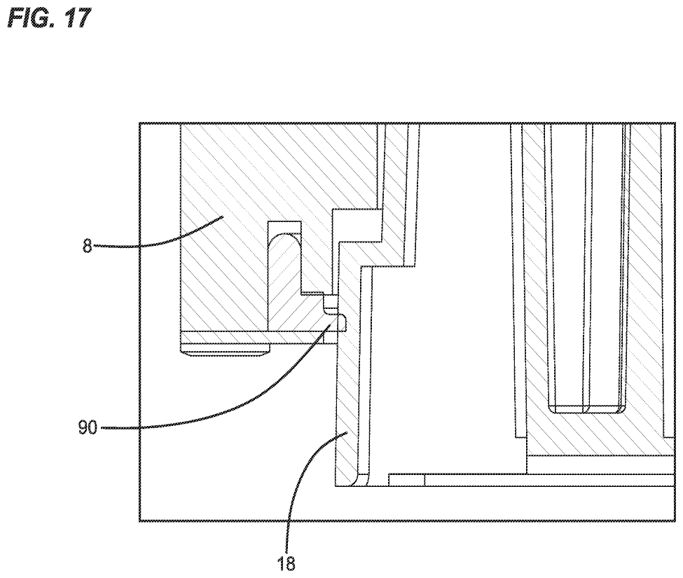

FIG. 17 is a cross-sectional view of the lid housing 8 and sample container 18. The lid housing 8 can further include a seal 90 that engages and compresses itself against an exterior sidewall of the sample container 18 to provide another seal between the lid housing 8 and sample container 18. The seal 90 can be an anaerobic seal that blocks gases from entering or escaping from the sample container 18 before the sample container is inserted into the cultivation chamber 104 of the microbioreactor 100. For example, there can be a need in some instances for an oxygen-free atmosphere inside the sample container 18, such that the container assembly 200 is configured for use as an anaerobic chamber. The seal 90 can prevent oxygen from entering the wells 61 of the sample container 18.

In view of the foregoing, a sample can be added to the sample container 18 in an anaerobic tent, and the sample container 18 can be sealed with the lid housing 8 before the container assembly 200 is taken to the microbioreactor 100, which may be located outside the anaerobic tent, for cultivation and/or further work. This is advantageous in that it allows users to work with the cultures freely without special equipment, and further advantageous in that the microbioreactor does not need to be positioned within the anaerobic tent and can thereby be more accessible. Accordingly, the anaerobic seal between the sample container 18 and lid housing 8 allows for easy transport of the container assembly 200 in an open-air environment.

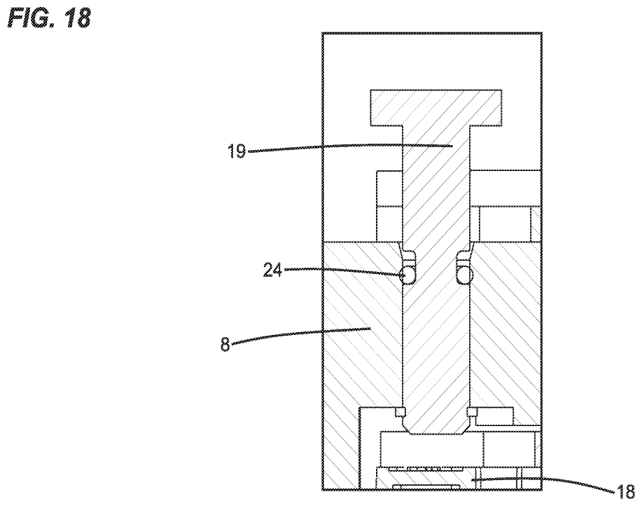

FIG. 18 is a cross-sectional view showing a release pin 19 of the container assembly 200. As shown in FIG. 18, the release pins 19 are each sealed by an O-ring 24, and can be used to release the lid housing 8 from the sample container 18. For example, the sample container 18 can be released from the lid housing 8 by holding the lid housing 8 and exerting pressure on the release pins 19 to push or eject the sample container 18 out of the lid housing 8.

FIGS. 19-21 show an example of the container assembly 200 that is configured for non-microfluidic applications. In this example, the container assembly 200 has two eccentric levers 7. FIG. 19 shows the container assembly 200 mounted to an orbital shaking platform 180, FIG. 20 shows a cross-sectional view of the container assembly 200 mounted to the orbital shaking platform 180 without connection to microfluidic gas channels 151, and FIG. 21 shows the orbital shaking platform 180 when the container assembly 200 is not mounted thereto.

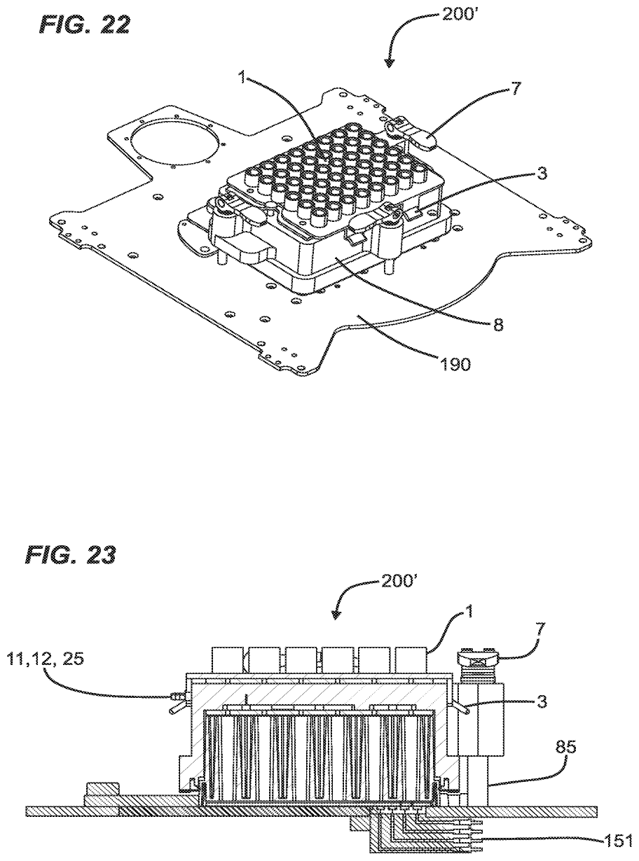

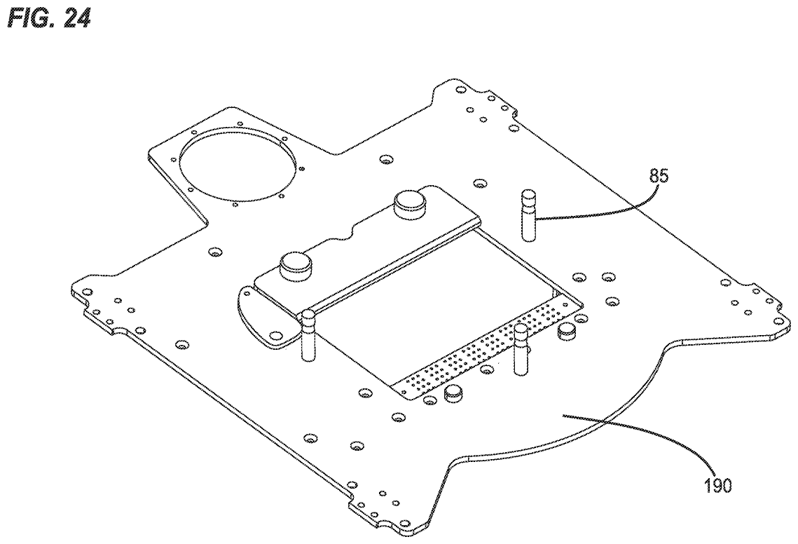

FIGS. 22-24 show views of an example of the container assembly 200′ that includes three gas ports 11, 12, 25 for microfluidic applications. FIG. 22 shows the container assembly 200′ mounted to an orbital shaking platform 190, FIG. 23 shows a cross-sectional view of the container assembly 200′ mounted to the orbital shaking platform 190 with connections to the microfluidic gas channels 151, and FIG. 24 shows the orbital shaking platform 190 when the container assembly 200′ is not mounted thereto. In this example, three eccentric levers 7 are provided on the container assembly 200′ because the levers are offset on the sides. By providing the third lever, pressure can be uniformly distributed around the entire assembly. Otherwise, the offset levers would not distribute pressure uniformly.

The microfluidic gas channels 151 are used for operating microfluidic valves that control the flow of reagents in the reservoir wells that are fed into the cultivation wells. The microfluidic gas channels 151 are used to pressurize the microfluidic valves such that the microfluidic valves are closed when pressure is applied to them and are opened when no pressure is applied. Through a specific order in which the microfluidic valves are opened and closed, a defined volume of reagents can be fed into the cultivation wells. In some examples, there are 96 microfluidic gas channels 151 which control each of microfluidic valve individually. This technology is described in more detail in U.S. Pat. No. 8,932,544, titled Microreactor Array, Device Comprising a Microreactor Array, and Method for Using a Microreactor Array, issued on Jan. 13, 2015, the entirety of which is hereby incorporated by reference.

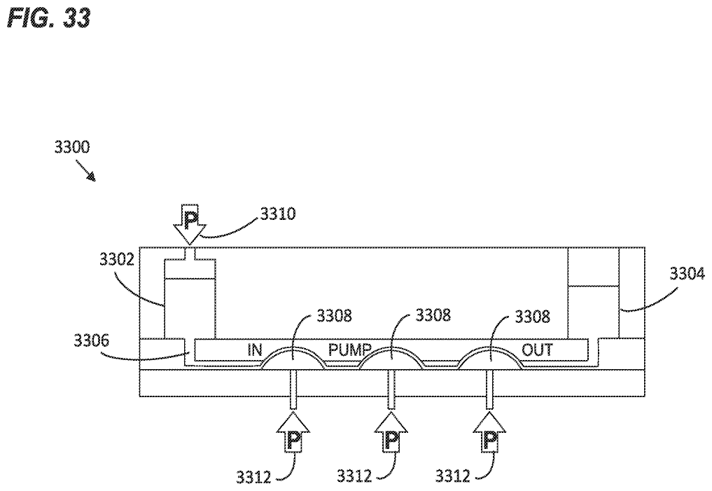

FIG. 33 illustrates a microfluidic valve configuration 3300 where a pressurizing gas 3310 pressurizes a headspace above a reservoir well 3302 causing liquid from the reservoir well 3302 to move down into a fluid duct 3306. A controlled sequence of feeding pressuring gas 3312 from the microfluidic gas channels 151 causes microfluidic valves 3308 along the fluid duct 3306 to open and close. The opening and closing of the microfluidic valves 3308 causes the liquid to move across the fluid duct 3306 until it reach the cultivation well 3304.

Microbial cultures require a gas atmosphere to grow. For most cells, oxygen is a critical component of the atmosphere. However, pure oxygen can be toxic to organisms such that it is often diluted with nitrogen to create an atmosphere like air with varying concentrations of oxygen. CO₂ from the atmosphere can be used to adjust the pH or as a carbon source for phototrophic organisms conducting photosynthesis. The container assembly 200 can be used to provide an atmosphere for the wells 61 having a mixture of air, oxygen, nitrogen, and CO₂.

The gases listed above can be mixed during an experiment. In some examples, two of the gases can be mixed. For example, to increase the oxygen concentration of the atmosphere, air can be mixed with oxygen. To decrease the oxygen concentration of the atmosphere, air can be mixed with nitrogen. To increase the CO₂ concentration of the atmosphere, air can be mixed with CO₂. In some examples, only two gases are mixed at a time. In alternative examples, more than two gases can be mixed.

In the above examples, the mixture is created by two or more valves controlled with a pulse-width-modulation (PWM) signal to set a ratio between the time each valve is opened or closed. The gas is fed through a gas inlet port (e.g., one of the gas ports 11, 12 in the example figures). The longer the time the valve is opened, higher the concentration of the gas that can pass the valve. After the gases are mixed, a sensor can measure the oxygen or CO₂ levels in the atmosphere. The control feedback is a controller that can adjust the PWM signal accordingly to reach the predefined values.

To avoid the loss of liquid in the sample container 18, the gas introduced into the container assembly 200 through the lid housing 8 can be saturated with humidity. This prevents evaporation of the medium in the wells 61 dedicated for cell cultivations. Therefore, the gas stream that is ultimately fed through one or more of the gas ports 11, 12, 25 may be led through a reservoir filled with water (or some other suitable liquid for humidifying the gas stream) at a suitable point along its flow (e.g., at some point between a source of the gas and the inlet gas port) so as to humidify the gas stream. For example, the gas stream that is fed into a gas inlet port (e.g., one of the gas ports 11, 12) may originate at one or more gas sources (e.g., gas canisters), can be mixed with another gas, and can then pass through a water reservoir, and ultimately be fed into the gas inlet port. In some examples, a tube may guide the gas stream to the bottom of the reservoir so that it has to pass through the water. In order to maximize the absorption of water in the reservoir, it is heated by a heat pad to a temperature set well above room temperature. In some examples, the additional gas port 25 (which feeds pressurizing gas into the space above the reservoir wells) may be humidified by a similar process. In other examples, the additional gas port 25 may not be humidified.

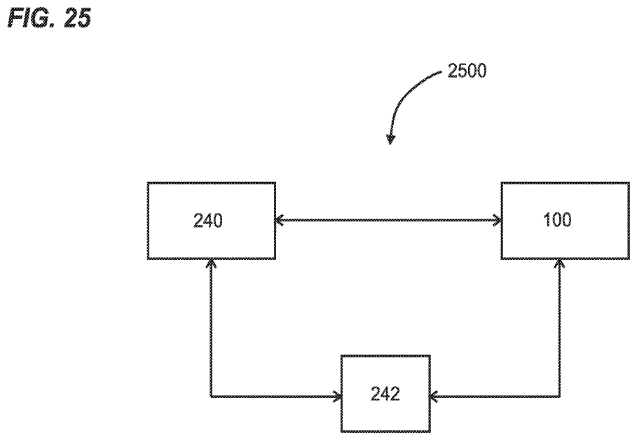

FIG. 25 schematically shows an example of a computer control system 2500 of the microbioreactor 100. As shown in FIG. 25, the computer control system 2500 includes a computer controller 240 that is operatively coupled to control the operations of the microbioreactor 100, as described above. The computer controller 240 is therefore operatively coupled to gas supplies, gas valves, sensors, actuators, and pipetting robot (collectively 242) in order to carry out the above-described functionalities. The computer controller 240 also comprises a computer storage medium that stores, in a tangible and non-transitory manner, a computer program product, that when executed by the computer controller 240, causes the computer controller 240 to carry out the above-mentioned functionalities.

Another embodiment includes at least one computer-readable medium storing data instructions that, when executed by at least one processing device (such as a processor of the computer controller 240), cause the at least one processing device to carry out one or more of the above-mentioned functionalities. For example, one embodiment includes at least one computer-readable medium storing data instructions that, when executed by at least one processing device, cause the at least one processing device to: sense measurement parameters associated with a sample container assembly having a gassing lid; process the sensed measurement parameters; and control a gas supply of at least one gas to the gassing lid based upon the processed measurement parameters.

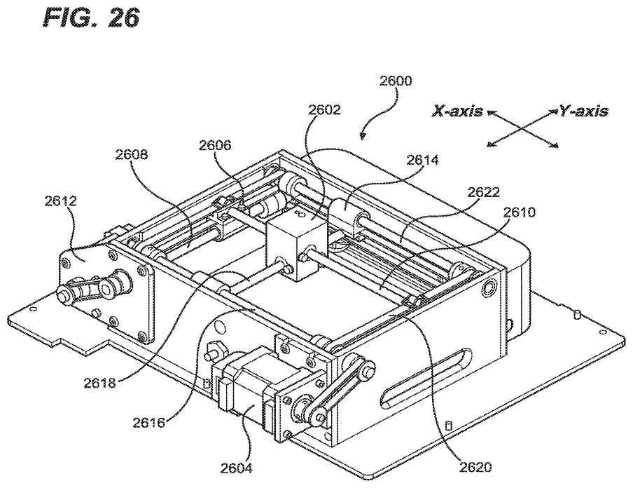

FIG. 26 is an isometric view of an example of a mechanical system 2600 of the microbioreactor 100. The mechanical system 2600 may be used by the microbioreactor 100 to position an optical sensor 2602 under the container assembly 200 when the container assembly 200 is held inside the cultivation chamber 104 of the microbioreactor 100.

The optical sensor 2602 is moved under each well 61 of the sample container 18, or under a subset of the wells 61 of sample container 18, to obtain measurements of the cell cultures in each well. The movement of the optical sensor 2602 is controlled by the mechanical system 2600 along two perpendicular axes (e.g., X and Y axes). Moving the optical sensor 2602 along the X and Y axes allows the optical sensor 2602 to be positioned under each well 61 of the sample container 18 to illuminate each well 61 with light, and to receive the scattered light that is returned back from the well 61 to obtain a measurement of one or more parameters such as biomass, pH, dissolved oxygen (DO), and fluorescence.

A first motor 2604 powers actuators 2606 to slide along shafts 2608 parallel to the Y-axis to control the position of the optical sensor 2602 along the Y-axis. The actuators 2606 carry a shaft 2610 that is parallel to the X-axis, and that is connected to the optical sensor 2602. The actuators 2606 are configured to move the optical sensor 2602 along a shaft 2618 to thereby control the position of the optical sensor 2602 along the Y-axis.

A second motor 2612 powers actuators 2614 to slide along shafts 2616 parallel to the X-axis to control the position of the optical sensor 2602 along the X-axis. The actuators 2614 are connected to the optical sensor 2602 via the shaft 2618 to move the optical sensor 2602 along the shaft 2610 to thereby control the position of the optical sensor 2602 along the X-axis.

In some examples, the first and second motors 2604, 2612 are step motors. In the example shown in FIG. 26, the first and second motors 2604, 2612 pull belts 2620, 2622 respectively to control the movement of the actuators 2606, 2614 along the perpendicular axes. Alternative examples are contemplated for moving the optical sensor 2602 along the perpendicular axes for positioning the optical sensor 2602 under each well 61.

FIG. 27 is an isometric view of a light-emitting diode array module (LAM) 2700 that can be used to illuminate the cultivation chamber 104 of the microbioreactor 100. The LAM 2700 can be an add-on module. The illumination from the LAM 2700 is similar to bright sunlight. The spectral composition of the light can be varied. The LAM 2700 allows for the high-throughput cultivation of phototrophic microorganisms within the microbioreactor 100.

The LAM 2700 includes a housing 2702. In some examples, the housing 2702 is made from aluminum. In some examples, the housing 2702 measures approximately 35 cm×26 cm×9.75 cm. Alternative materials and size measurements for the housing 2702 are possible.

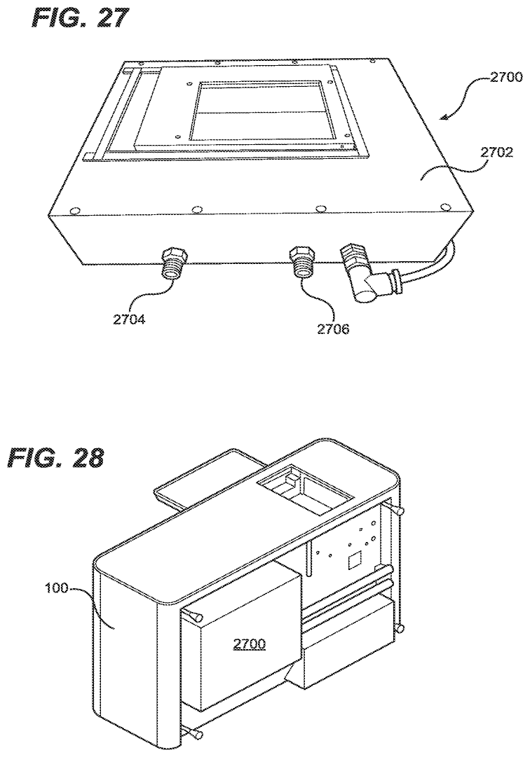

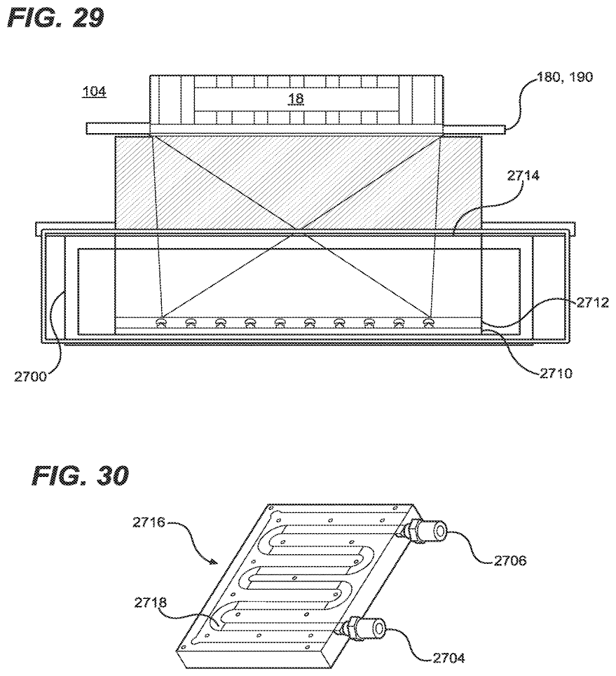

FIG. 28 is a bottom isometric view of the LAM 2700 mounted underneath the microbioreactor 100. FIG. 29 is a schematic diagram of the LAM 2700. Referring now to FIGS. 28 and 29, the LAM 2700 is configured to homogeneously illuminate the bottom of the sample container 18 (e.g., a microplate or microtiter plate) placed on an orbital shaking platform 180, 190 (e.g., a shaker) inside the cultivation chamber 104 of the microbioreactor 100.

The LAM 2700 includes an array of light-emitting diodes (LEDs) 2710 that emits the light to the illuminate the cultivation chamber 104, and can include a lens 2712 to focus the light and a transparent quartz plate 2714 that allows the light to pass through, and that protects the internal components of the LAM 2700 including the arrays of LEDs 2710 and lens 2712.

The LED 2710 can generate a considerable amount of heat over time. Thus, the LAM 2700 includes a cooling plate 2716 (see FIG. 30) that can be used to cool down the LAM 2700 and/or the cultivation chamber 104 of the microbioreactor 100.

FIG. 30 is an isometric view of the cooling plate 2716. Referring now to FIGS. 27 and 30, the cooling plate 2716 includes an inlet 2704 that receives a liquid coolant (e.g., water) that runs through a coil 2718 to cool down the LAM 2700 before exiting through an outlet 2706. The coil 2718 can have a serpentine shape to increase the surface area of the coil 2718 and thereby increase the cooling effect of the liquid coolant that runs through it.

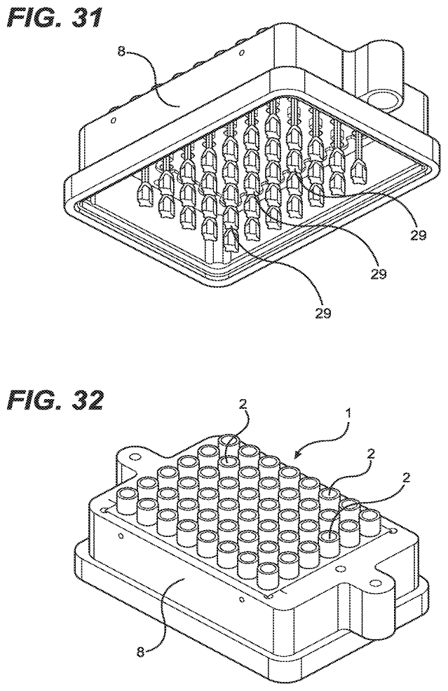

FIG. 31 is a bottom isometric view of an example of the lid housing 8 that is adapted to cool the sample container 18 (e.g., a microplate or microtiter plate). FIG. 32 is a top isometric view of the lid housing 8. In this example, the lid housing 8 (e.g., gassing lid) includes cooling pins 29 that connect to the guide elements 2 on the one end, and that extend downward into gaps 62 between the wells 61 of the sample container 18 (see FIG. 8). The cooling pins 29 and guide elements 2 can be made of conductive materials and can thus act as a heat sink for taking heat away from the sample container 18 and diffusing it to ambient air outside of the container assembly 200. In some examples, to dissipate the heat more quickly and to improve the thermal contact between the cooling pins 29 and sample container 18, the gaps 62 between the wells 61 of the sample container 18 can be filled with a liquid, such as demineralized water.

Thus, the cooling pins 29 can create a heat exchange between the sample container 18 and well-tempered air in the upper portion of the cultivation chamber 104, while at the same time serves as a guide for the pipette tip 71. The cooling pins 29 can maintain the temperature inside the sample container 18 at an acceptable level, and that is uniformly distributed within the sample container 18. Also, the cooling pins 29 can help to maintain the temperature inside the upper and lower portions of the cultivation chamber 104 at an acceptable level.

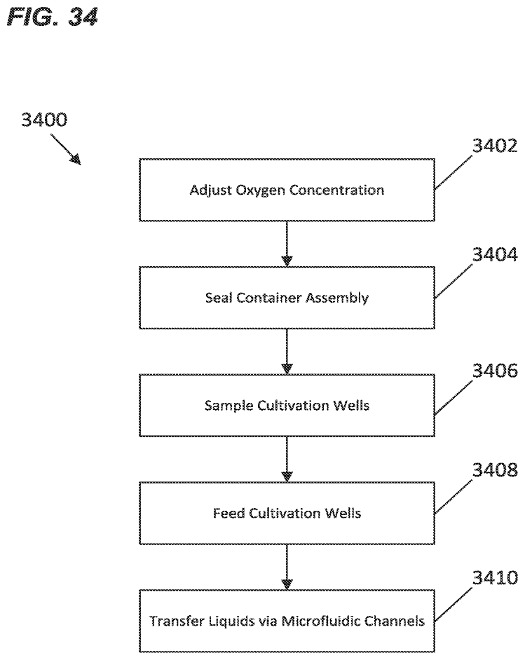

FIG. 34 illustrates an example of a method 3400 of anaerobic cultivation that can be performed using the container assembly 200. The method 3400 includes an operation 3402 of adjusting oxygen concentration in the headspace above the cultivation wells to a predetermined oxygen range that is below a threshold amount (e.g., between 0%-5%, 0%-10%, any range in between) while in the anaerobic environment. Next, the method 3400 includes an operation 3404 of sealing the container assembly 200. In accordance with the examples described above, the container assembly 200 can be sealed using the eccentric levers 7.

The method 3400 next includes an operation 3406 of sampling from cultivation wells through the various layers and components of the container assembly 200. For example, a pipette tip 71 can be inserted through a guide element 2, through an aperture 6 of the second resilient layer 4, through a through-hole 23 of the lid housing 8, and through an aperture 15 of the first resilient layer 13, such that the pipette tip 71 can pierce the sterile layer 16, and obtain a sample from a cultivation well of the sample container 18. In some examples, the pipette tip 71 can be operated by the pipetting robot 70. Alternatively, the pipette tip 71 can be operated by hand. Operation 3406 can be performed while the container assembly 200 maintains the anaerobic atmosphere in the headspace above the cultivation well, as set by operation 3402.

In some examples, the method 3400 can include an operation 3408 of adding reagents, media, or pH to the cultivation wells through the various layers and components of the container assembly 200. For example, a pipette tip 71 can be inserted in accordance with the description provided above with respect to operation 3406 for adding reagents, media, or pH to the cultivation wells. Operation 3406 can be performed while the container assembly 200 maintains the anaerobic atmosphere in the headspace above the cultivation well.

In some examples, the method 3400 can include an operation 3410 of feeding liquids such as reagents, media, or pH adjustment solution via integrated microfluidics from reservoir wells to cultivation wells to feed the cultivation wells or adjust the pH in the cultivation wells. Operation 3410 can be performed in examples where the sample container 18 is integrated with microfluidics such as on the orbital shaking platform 180, 190.

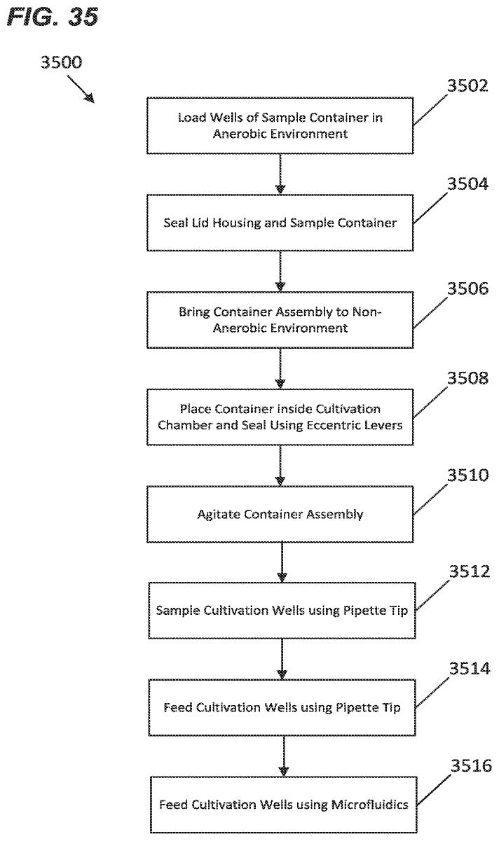

FIG. 35 illustrates another example of a method 3500 of anaerobic cultivation that can be performed using the container assembly 200. The method 3500 includes an operation 3502 of loading the wells 61 of the sample container 18 (e.g., microtiter plate or microplate) with cells and media in an anerobic environment. In some examples, the anerobic environment is an anaerobic tent that has a very low oxygen concentration.

Next, the method 3500 includes an operation 3504 of sealing the lid housing 8 onto the sample container 18 using the seal 90. As described above, the seal 90 prevents oxygen from entering the wells 61 of the sample container 18.

Next, the method 3500 includes an operation 3506 of bringing the container assembly 200 outside the anerobic environment to a non-anaerobic environment. In some examples, the non-anaerobic environment refers to an environment that is outside of the anaerobic tent such as the normal environment of a lab. In some examples, the non-anaerobic environment is where the microbioreactor 100 is located.

Next, the method 3500 includes an operation 3508 of placing the container assembly 200 into the cultivation chamber 104 of the microbioreactor 100 and sealing the container assembly using the sealing mechanism 9 with the eccentric levers 7.

Next, the method 3500 includes an operation 3510 of continuously or semi-continuously agitating the container assembly 200 inside the cultivation chamber 104. For example, the container assembly 200 can be agitated by motion of the orbital shaking platform 180, 190 on which the container assembly 200 is seated or attached.

Next, the method 3500 can include an operation 3512 of sampling the cultivation wells inside the container assembly 200 with a pipette tip 71 such as by removing some of the liquid in the cultivation wells. Operation 3512 can be performed while the container assembly 200 is being agitated (see operation 3510). In some examples, the pipette tip 71 is operated by the pipetting robot 70. Alternatively, the pipette tip 71 can be operated by hand, either singly or using a multi-pipette tool. Operation 3512 can be similar to operation 3406 described above with respect to the method 3400.

Next, the method 3500 can include an operation 3514 of feeding the cultivation wells with reagents, nutrients, or media with the pipette tip 71. Operation 3514 can be performed while the container assembly 200 is being agitated (see operation 3510). In some examples, the pipette tip 71 is operated by the pipetting robot 70. Alternatively, the pipette tip 71 can be operated by hand. Operation 3514 can be similar to operation 3408 described above.

Next, the method 3500 can include an operation 3516 of feeding the cultivation wells with reagents, nutrients, or media via integrated microfluidics (e.g., the described pneumatic valve system at the bottom of the sample container 18). Operation 3516 can be similar to operation 3410 described above with respect to the method 3400.

Probiotics are living bacteria that have health-promoting benefits and bio-functional effects on the human organism. They are commonly used to increase the number of desirable bacteria in the intestine and to regenerate the intestinal flora, for example after antibiotic treatments. That is one reason why the market for probiotics or probiotic nutritional supplements has greatly increased in value. The research field of the human intestinal microbiome and its health-promoting benefits is particularly important for the nutrition industry. Therefore, scientific research on anaerobic or microaerophilic cultivation techniques, such as the cultivation of probiotics under microbiome-like conditions, is essential. Probiotics include a whole range of anaerobic bacteria such as Lactobacillus or Bifidobacterium. Among the various probiotic bacteria, Bifidobacterium spp. is one of the most widely used and studied probiotic bacterium species. They are classified as strict anaerobes due to the incapability of oxygen respiration and growth under aerobic cultivation conditions, and they are a major member of the dominant human gut microbiota. They play a significant role in controlling the pH through the release of lactic and acetic acids, which restrict the growth of many potential pathogenic bacteria. In the intestinal tract of breast-fed infants, Bifidobacterium is the predominant cell species. It accounts for more than 80% of microorganisms in the intestine. There are more than 200 known species of Lactobacillus, the largest and most diverse genus within the lactic acid bacteria which that is generally recognized as safe (GRAS) by the US Food and Drug Authority Administration (FDA). Lactobacillus spp. have been deployed and studied extensively as fermentation starter cultures for dairy products or probiotics due to their applied health potential.

In this application, anaerobic cultivation experiments can be performed using the container assembly 200 which includes the sample container 18 in combination with the gassing lid. The container assembly 200 is a bench-top device for high-throughput screening of microbial cultivations that enables online-monitoring of the most common cultivation parameters such as biomass, pH value, oxygen saturation of the liquid phase (DO) and fluorescence intensity of various fluorescing molecules or proteins. To achieve high throughput, cultivations are carried out in SBS/SLAS standard format microtiter plates (e.g., the sample container 18) with 48 wells each, which allows for the simultaneous run of up to 48 batches in the container assembly 200. Furthermore, the simplicity of using the gassing lid to perform anaerobic batch and fed-batch cultivations of the probiotic bacteria Lactobacillus casei, Lactobacillus plantarum, and Bifidobacterium bifidum. A main advantage of the gassing lid is that feeding and pH control can now take place simultaneously during direct nitrogen (e.g., 100% N2) gassing of the sample container 18 with adjustable flowrates between 5-50 mL/min.

### Anaerobic Cultivations of Lactobacillus Strains

All cultivations of Lactobacillus spp. (Lactobacillus casei DSM 20011 or Lactobacillus plantarum DSM 20174) took place in MRS broth at 37° C. ambient temperature and under anaerobic conditions. MRS broth was enriched with 0.5 g/L cysteine-HCl which serves as reducing agent for oxidation-reduction potential by reducing the residual molecular O₂ in the medium. All precultures were performed in a 250 mL Erlenmeyer flask. For this purpose, 20 mL of prepared MRS broth was inoculated with 1 mL cryoculture and then cultivated for at least 24 hours under anaerobic conditions. The main culture was then set to ODₛₜₐᵣₜ=1 in MRS broth. Subsequent microbioreactor cultivations were performed in a microfluidic round well plate for pH-controlled batch and fed-batch cultivations. The cultivations were conducted at 37° C., 600 rpm and enabled humidity control. The start volumes of the cultivation wells were set to 2,000 μL and the maximum volumes to 2,400 μL. Online monitoring of biomass (gain 3) and the measurement of pH (LG1) and dissolved oxygen DO (RF) were performed by the microbioreactor 100. A more detailed overview of the fed-batch cultivation conditions of L. casei are shown in table 1.

TABLE 1
Fed-batch cultivation conditions for L. casei

| | CONTENT | MICROFLUIDIC SETTINGS |
|---|---|---|
| RESERVOIR A | 500 g/L | Pump volume: 0.16 μL |
| (FEED) | glucose | Filling volume: 1,900 μL |
| | | Feed-start: >7.5 h or >10 h |
| | | Constant feed: 4 μL/h |
| RESERVOIR B | 3M NaOH | Pump volume: 0.30 μL |
| (PH-CONTROL) | | Filling volume: 1,900 μL |
| | | pH-control-start: >0.5 h |
| | | PI settings: MEDIUM |
| CULTIVATION | L. casei in | Start volume: 2000 μL |
| WELLS | MRS broth | Max. volume: 2400 μL |
| | | pH-control: pH 6.0 |

Anaerobic Cultivations of B. bifidum in the Microbioreactor

All cultivations of Bifidobacterium bifidum were performed in MRS broth at 37° C. and under anaerobic conditions. MRS broth was enriched with 0.5 g/L cysteine-HCl which serves as reducing agent for the oxidation-reduction potential by reducing the residual molecular O₂ in the medium. The preculture cultivations took place in a 250 mL Erlenmeyer flask. For this purpose, 20 mL MRS broth was inoculated with the content of one capsule and then cultivated for at least 24 h at 37° C. under anaerobic conditions. The main culture was set to ODₛₜₐᵣₜ=1.0 in MRS broth.

For the main culture in the container assembly 200, pH-controlled batch and fed-batch cultivations at 37° C., 600 rpm, enabled humidity control, online monitoring of biomass (gain 3), pH (LG1), and DO (RF) were performed. A more detailed overview of the fed-batch cultivation conditions of B. bifidum are listed in table 2.

TABLE 2
Fed-batch cultivation conditions for B. bifidum

| | CONTENT | MICROFLUIDIC SETTINGS |
|---|---|---|
| RESERVOIR A | 500 g/L | Pump volume: 0.16 μL |
| (FEED) | glucose | Filling volume: 1,900 μL |
| | | Feed-start: >5 h |
| | | Constant feed: 4 μL/h |
| RESERVOIR B | 3M NaOH | Pump volume: 0.30 μL |
| (PH-CONTROL) | | Filling volume: 1,900 μL |
| | | pH-control-start: >0.5 h |
| | | PI settings: MEDIUM |
| CULTIVATION | B. bifidum in | Start volume: 2000 μL |
| WELLS | MRS broth | Max. volume: 2400 μL |
| | | pH-control: pH 6.0 |

### Layout Settings in the Sample Container 18:

All fed-batch cultivations took place in the sample container 18 (FIG. 8). Row A contained 1,900 μL of the glucose feed solution and row B was filled with 1,900 μL of the pH-adjusting agent. Software adjusted the pump volumes to 0.30 μL for aqueous solutions (3 M NaOH) and to 0.16 μL for the more viscous feed solution (500 g/L glucose).

In all fed-batch experiments, the feeding was time triggered and the feed profile was set to a constant feed with 4 μL/h. The pH control was set to pH 6.0. The anaerobic conditions during all cultivations in the sample container 18 were achieved by using the gassing lid, which was attached to the sample container 18 after it was prepared and sealed with the gas permeable sterile silicone foil (F-GPRSMF32-1).

### Results

Fed-Batch Cultivation of Lactobacillus casei in the Microbioreactor

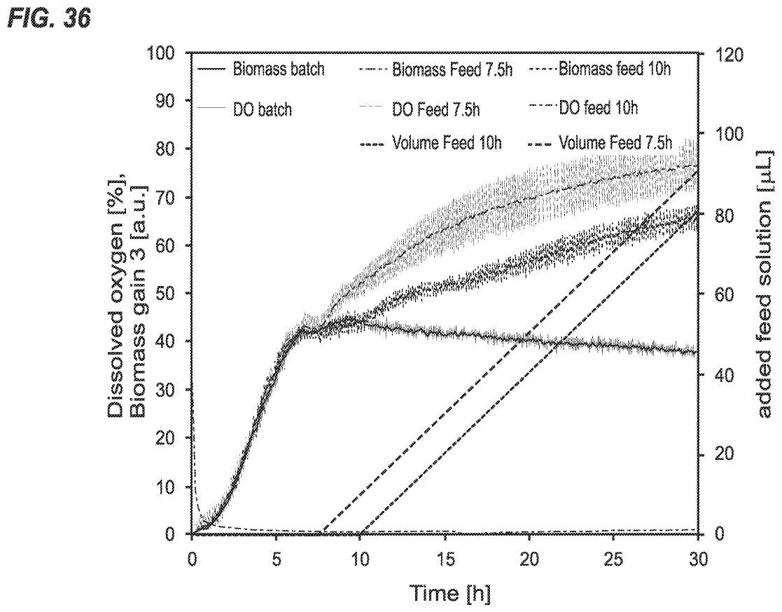

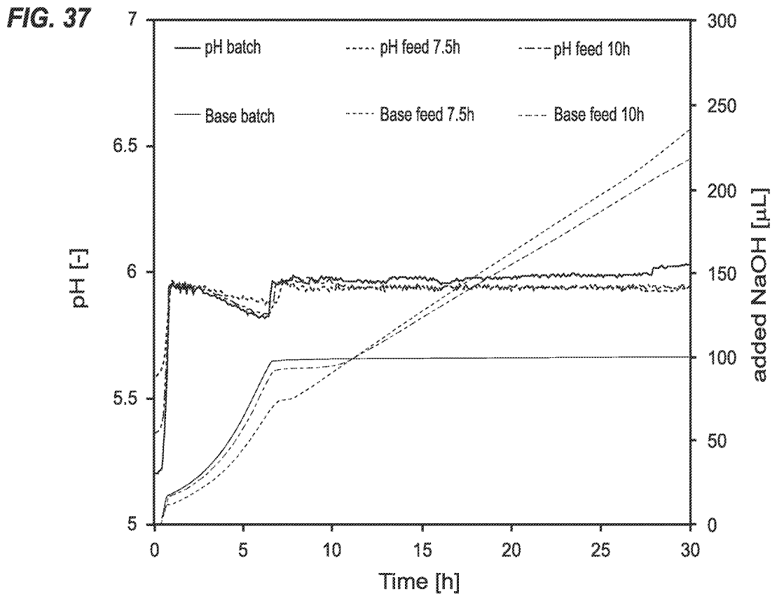

In FIGS. 36 and 37, the cultivation process of Lactobacillus casei in MRS broth is shown. In FIG. 36, the online signals of biomass and dissolved oxygen (DO) signal, and the volume of the added feed solution (500 g/L glucose) are presented. In FIG. 37, the online values of pH and the associated volumes of NaOH are plotted against cultivation time.

Here, three different process setups were applied: a batch cultivation and two fed-batch cultivations. One with a feed start after 7.5 hours and the other with a feed start after 10 hours. With a continuous flowrate of 30 mL/min N₂, the DO decreased steadily. After 45 minutes, a DO below 5% was reached and decreased further. After 4.5 hours the DO reached below 0.5% and continued to drop towards 0%. With the initiation of the stationary phase of the culture at around 6.7 hours, the exponential growth stops, and the biomass signal was 42 a.u. in all three culture approaches at this timepoint. The batch culture grows further slowly to a maximum of 44 a.u. at 9.5 hours then it steadily decreases to a final biomass signal of 38 a.u. at the end of cultivation. An increase in the biomass signal is correlated with the addition of the feed solution. As soon as the feed starts, an increase of the biomass signal is visible. The final biomass signal for the 7.5 h-fed-batch process was 76.3 a.u. and for the 10 h-fed-batch process, it led to a final biomass signal of 65.5 a.u. after 30 hours. The values for the added base solution are growth correlated. The addition of 3 M NaOH was stopped with the initiation of the stationary phase because no further bacterial acid production took place due to no growth. In the case of the constant addition feed solution, the acid production continued and thus, base was further needed to maintain the pH set point value of pH 6.0.

This experiment shows that the container assembly 200 is a suitable device for anaerobic cultivations due to the gassing lid and the successful application of pH-control and feeding at the same time with direct anaerobic gassing.

### Technical and Biological Validation of the Anaerobic Conditions in the BioLector XT Device

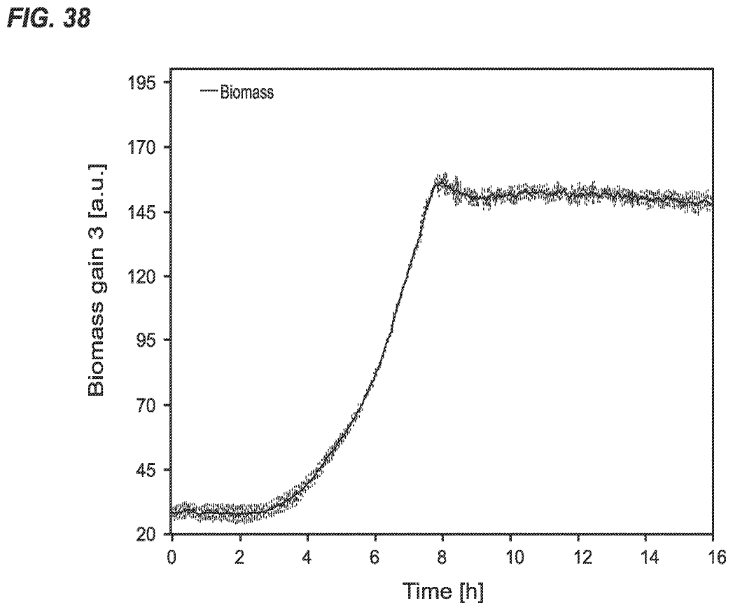

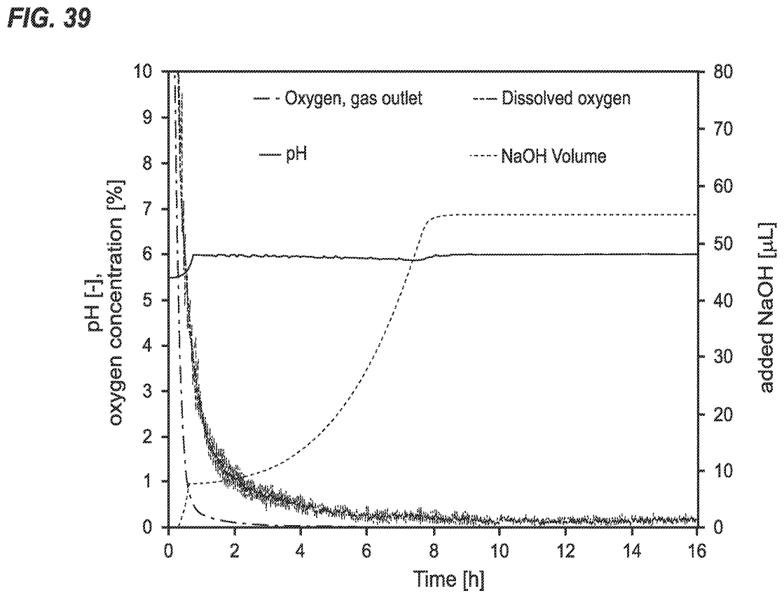

Maintaining anaerobic conditions during the whole cultivation time is an important requirement in case of the cultivation of oxygen sensitive organisms. In the following experiment, an external oxygen sensor was installed at the gas outlet of the container assembly 200 to validate the technical functionality of the gassing lid and to prove the tightness of the gassing lid and thus, the anaerobic atmosphere. In FIGS. 38 and 39, the experimental data of a batch cultivation of Lactobacillus plantarum (L. plantarum) are shown. In FIG. 38, the online biomass signal (gain 3) is shown. In FIG. 39, the online signal of dissolved oxygen in the culture broth and the oxygen concentration in the gas outlet of the container assembly 200, the online pH signal and the added NaOH volume for the pH-control are shown.

After a lag-time of 2.86 hours, the exponential growth started. The final biomass signal was 155.865 a.u. (OD600=9.01±0.07) after 7.96 hours when the stationary phase was initiated. During the growth of L. plantarum, lactic acid production took place. That acid formation growth is correlated to the added NaOH volume to maintain pH 6. With a continuous flowrate of 30 mL/min N₂ the DO decreased steadily. After 39 min, a DO below 5% was reached and decreased further. After 4 hours, the DO dropped further below 0.5% and continued to drop towards 0%. The external sensor showed a final oxygen concentration of 0.029% after a cultivation time of 16 hours.

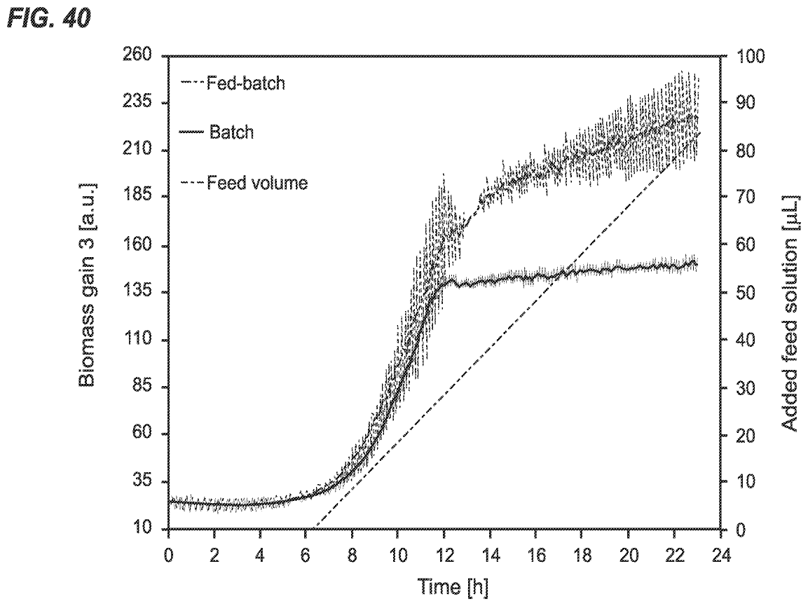

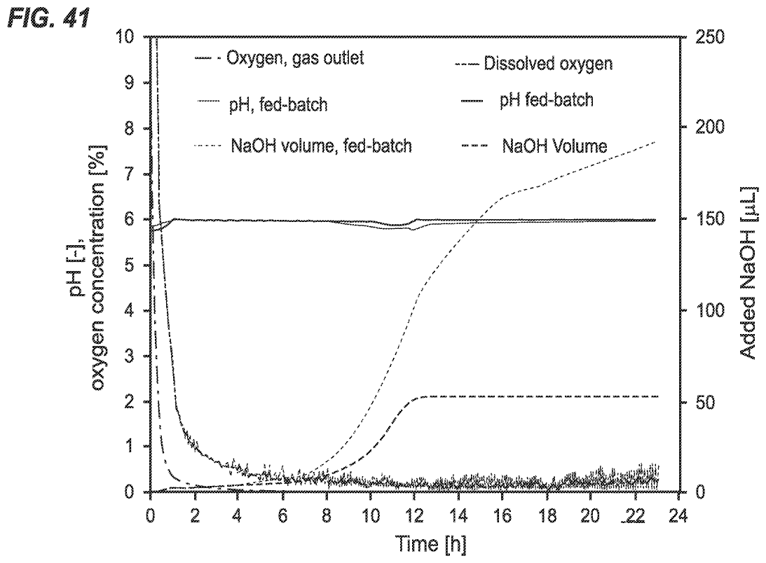

With this cultivation example, the technical functionality was validated, but the fact that Lactobacillus spp. can also grow under aerobic conditions and can even metabolize oxygen is not sufficient evidence for the biological validation of anaerobic cultivation in the container assembly 200. Therefore, the strict anaerobic Bifidobacterium bifidum was cultivated. The successful cultivation of this strain serves as the biological validation for anaerobic cultivations in the container assembly 200. In FIGS. 40 and 41, experimental data of a batch as well as a fed-batch cultivation of B. bifidum is shown. In FIG. 40, online signals of biomass and the added feed volume is plotted against the cultivation time. In FIG. 41, the online (optodes) signal of pH and DO, as well the added volume of 3 M NaOH and the oxygen signal of the external gas sensor in the gas outlet of the container assembly 200, are presented.

After a lag-time of 2.4 hours, the exponential growth started and for the batch culture the biomass signal reached a final value of 147.57 a.u. (OD600=8.3±0.57). In contrast to the batch culture, an extended exponential growth phase is observable. This phenomenon is caused by a higher amount of glucose in the medium as the feed already started after 6 hours. After 23 hours of cultivation, a maximum biomass value of 227.3 a.u. (OD600=15.93±0.69) was achieved. During the growth of B. bifidum, lactic acid production occurred and its growth correlated, which is observable in the curve of the addition of NaOH to maintain the pH at pH 6. In total, 193.56 μL of 3 M NaOH were pumped into the culture broth. With a continuous flowrate of 30 mL/min N2, the DO decreased steadily.

The external oxygen data already described for the first 16 hours since the cultivation of L. plantarum (as described earlier) and B. bifidum were gained simultaneously in the same container assembly 200 run and thus, the sample container 18, gassing lid, and external gas sensor were used. It is observable that the DO signal slightly increases from 18 hours, which could be explained by the technically conditioned signal drift of the oxygen optodes with a drift at 0% oxygen of <0.5% O2 per day. The data of the external oxygen sensor showed a value of 0.029% oxygen in the gas outlet of the container assembly 200 after 23 hours, confirming that the anaerobic cultivation conditions were maintained over the entire cultivation time.

In conclusion, a successfully conducted cultivation experiment of an anaerobic organism in the container assembly 200 is shown. In combination with microfluidic chip technology and the direct nitrogen gassing via the gassing lid, the simultaneous performance of pH control, feeding, and direct nitrogen gassing can be performed in small scale cultivations.

To sum up, the technical and biological validation of the cultivation of probiotics like Lactobacillus spp. and Bifidobacterium bifidum in the container assembly 200 in combination with the anaerobic gassing lid are shown. The microfluidic chip technology combined with the direct nitrogen gassing of the sample container 18 via the gassing lid enables the simultaneous performance of pH control, feeding and direct nitrogen gassing in small scale cultivation systems. It is a suitable system for the cultivation of anaerobic bacteria.

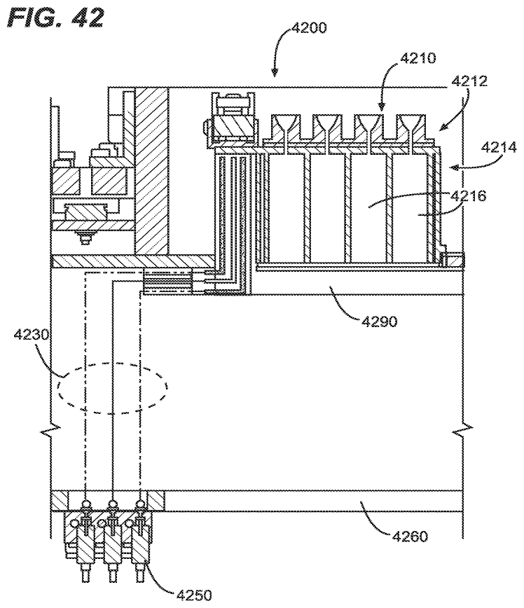

FIG. 42 is a cross-sectional view of an example bioreactor system 4200 that allows for precise gas control and air-tight sealing of individual reservoirs (or in some embodiments, subsets of reservoirs). The bioreactor system 4200 enables analysis of cellular responses to external stimuli. In particular, the bioreactor system 4200 uses microfluidics for cell culturing with a high degree of control over process variables. Moreover, as will be described in greater detail below, the bioreactor system 4200 enables reservoir-specific regulation of gas atmosphere (e.g., some combination of nitrogen, oxygen, and carbon dioxide) in the headspace of each cell culture. This allows for precise control of gas conditions in each reservoir without contamination from neighboring reservoir gases. Although the example embodiments described below focus on gas control and sealing of individual reservoirs, the disclosure contemplates that other embodiments may have gas control and sealing of subsets of reservoirs (e.g., subsets of two or more reservoirs).

The bioreactor system 4200 includes one or more sample container assemblies 4210. Each sample container assembly 4210 includes a microfluidic lid assembly 4212 and a sample container 4214. Similar to that previously described, the sample container 4214 includes rows of reservoirs 4216, or wells, each configured to separately contain a cell culture or reagent. Additionally, though the sample container 4214 is covered by the microfluidic lid assembly 4212 it remains accessible for one or more pipette tips to probe the reservoirs 4216. The sample container assembly 4210 thus allows a pipette tip to enter the sample container 4214 while preventing gas from escaping.

In addition to creating an air-tight seal above the sample container 4214, the microfluidic lid assembly 4212 seals off each reservoir 4216. Furthermore, as will be described in greater detail below, the microfluidic lid assembly 4212 includes a structure which enables individually controlling the gas concentrations in the headspace of each reservoir 4216 by introducing/removing gases via gas tubes 4230. The amount of each gas introduced into each reservoir headspace is controlled by a valve array 4250 coupled with the gas tubes 4230. The valve array 4250 may be attached to a base plate 4260 of the bioreactor system 4200 disposed below a shaking table 4290. Similar to that previously described with respect to orbital shaking platform 180/190, the shaking table 4290 is configured to couple with and/or move the sample container assembly 4210 for cell culture experiments. Accordingly, the gas tubes 4230 may comprise a flexible material and/or be arranged in an S-shape to allow bending in the gas tubes 4230 to withstand the shaking motion without interfering with it.

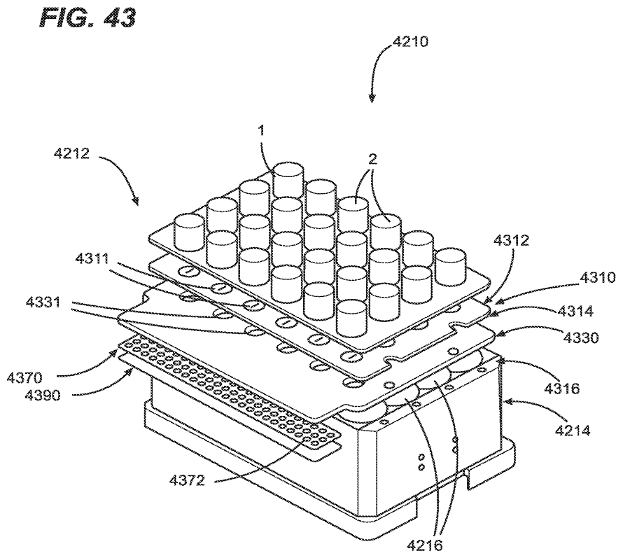

FIG. 43 is a perspective exploded view of the sample container assembly 4210. In particular, FIG. 43 shows an example arrangement of components of the microfluidic lid assembly 4212 including a guide structure 1 with guide elements 2 disposed thereon, a resilient layer 4310 disposed underneath the guide structure 1, and a microfluidic structure 4330 disposed underneath the resilient layer 4310. The resilient layer 4310 may comprise a panel of resilient polymer material, such as a silicone layer, that is situated between the guide structure 1 and microfluidic structure 4330.

In some embodiments, each side of the resilient layer 4310 may be provided with an adhesive layer 4312/4314, an adhesive coating or layer, or some other suitable adhesive application. In other embodiments, some other means of fastening the resilient layer may be used (e.g., a screw, clasp, or some other fastener). Accordingly, the guide structure 1 and one or more guide elements 2 attach, couple, or adhere with a top surface of the resilient layer 4310, and the microfluidic structure 4330 attaches, couples, or adheres with a bottom surface of the resilient layer 4310. Advantageously, components of the microfluidic lid assembly 4212, including the guide structure 1, guide elements 2, resilient layer 4310, and/or microfluidic structure 4330, may form layers of an integral unit that is disposable (e.g., for single-use application).

With the microfluidic lid assembly 4212 assembled and/or placed on top of the sample container 4214, the microfluidic structure 4330 is disposed over the reservoirs 4216 to create a seal along an outside perimeter of the sample container 4214. The resilient layer 4310 includes slits 4311 or apertures that align with respective reservoirs 4216 (as described in greater detail with respect to resilient layers 13 and 4).

Similarly, and as described in greater detail below, the microfluidic structure 4330 includes through-holes 4331 having corresponding alignment with the slits 4311 and reservoirs 4216. This arrangement advantageously seals each reservoir 4216 while still enabling a pipette tip to insert through a corresponding slit 4311 and through-hole 4331 to access a reservoir 4216.

In some embodiments, the microfluidic lid assembly 4212 is configured to be adhered to the sample container 4214 with an adhesive 4316. In one embodiment, a bottom surface of the microfluidic lid assembly 4212 (or bottom surface of the microfluidic structure 4330) is adhered to a top surface of the sample container 4214. In some embodiments, the adhesive 4316 comprises an ultraviolet curing adhesive to attach, couple, adhere, and/or seal an upper face of the outside perimeter of the sample container 4214 with a bottom face of the microfluidic lid assembly 4212 (or bottom face of the microfluidic structure 4330). The sample container assembly 4210 may thus form a single sealed unit that allows cell cultivation within each reservoir 4216. Advantageously, the sample container assembly 4210 is suitable for applications with high sterility demands such as mammalian cell culture since it may be formed as an integrated, disposable device that allows for increased sterility. The sample container assembly 4210 is also suitable for mammalian cell culture since it allows for precise individual well control (e.g., pH and gases).

The microfluidic structure 4330 is configured to receive gases from the gas tubes 4230 via gas inlets (not shown in FIG. 43) on its bottom surface. The gas reception portion or area of the microfluidic structure 4330 extends outside the structural body of the sample container assembly 4210 (or outside a perimeter of the sample container 4214) in assembled form. In some embodiments, at this area of the microfluidic structure 4330, the bottom surface is provided with an adhesive member 4370 to attach, couple, or adhere a sterile filter 4390 thereto. The sterile filter 4390 is configured to sterilize gases entering the microfluidic structure 4330 to prevent contamination of the gases. The adhesive member 4370 may include gas openings 4372 for allowing gases from corresponding gas tubes 4230 into the microfluidic structure 4330. In one embodiment, the sterile layer 4390 comprises a gas-permeable film (e.g., a plastic film) with pores that are configured to allow gas molecules to pass therethrough while filtering out microbes (e.g., via 200 nm pores).

The microfluidic structure 4330 and the sample container 4214 may be adhered such that they form an air-tight seal. These components may be manufactured such that they leave no gaps when they are adhered together, thus forming an air-tight seal. In this manner, each well of the sample container 4214 may be individually sealed by the microfluidic structure 4330. In other embodiments, a gasket or other sealing element may be placed in between the microfluidic structure 4330 and the sample container 4214 so as to form an air-tight seal.

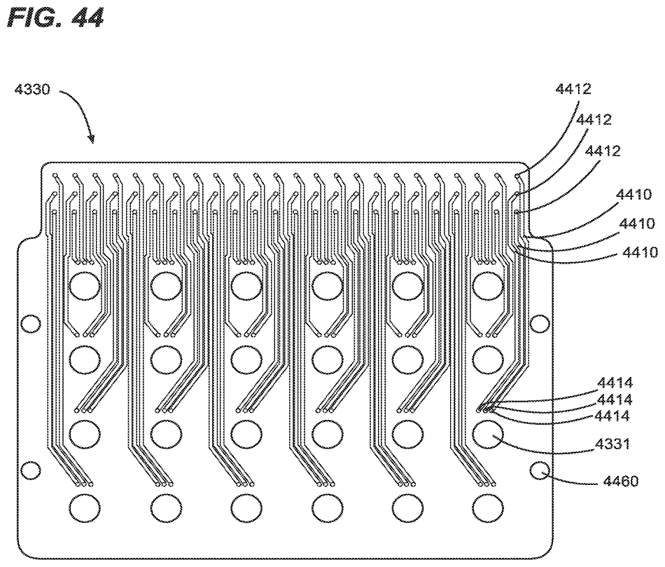

FIG. 44 is a cross-sectional view of the microfluidic structure 4330. FIG. 44 shows the microfluidic structure 4330 includes microfluidic channels 4410 each configured to transport gas from a gas inlet 4412 to a gas outlet 4414. The gas inlets 4412 comprise openings in the bottom surface of the microfluidic structure 4330 and are configured to receive gases via corresponding gas tubes 4230. The gas outlets 4414 comprise openings in the bottom surface of the microfluidic structure 4330 and are configured to provide gases to corresponding reservoirs 4216. The microfluidic structure 4330 is fixed above the reservoirs and is thus configured to seal each of the reservoirs 4216 individually while supplying each reservoir 4216 with a precise amount of one or more gases via the microfluidic channels 4410.

Suppose, for example, the microfluidic structure 4330 is configured to provide a precise combination of three gases (e.g., nitrogen, oxygen, and carbon dioxide) to each of a total of twenty-four reservoirs 4216. The microfluidic structure 4330 thus includes twenty-four through-holes 4331 to align with the headspace of the reservoirs 4216. Additionally, in this example, the microfluidic structure 4330 includes three microfluidic channels 4410 for each through-hole 4331 or reservoir 4216 (e.g., one channel each for nitrogen, oxygen, and carbon dioxide). Accordingly, the microfluidic structure 4330 includes seventy-two microfluidic channels 4410 (with corresponding gas inlets 4412 and gas outlets 4414) and is configured to provide an independent control of nitrogen, oxygen, and carbon dioxide to each reservoir 4216. It will be appreciated, however, that alternative configurations for a different number of reservoirs 4216 and/or combinations of gases for each reservoir 4216 are contemplated.

The microfluidic structure 4330 may also include one or more indexing holes 4460 to facilitate alignment with other components of the sample container assembly 4210. Generally, with the microfluidic structure 4330 coupled or aligned with the sample container 4214, the gas inlets 4412 are disposed outside the perimeter of the sample container 4214 and the gas outlets 4414 are disposed inside the perimeter of the sample container 4214. A subgroup of microfluidic channels 4410 that transport gas to the same reservoir 4216 may be disposed adjacently with each other in a plane of the microfluidic structure 4330. The subgroup of microfluidic channels 4410 may also merge together and/or terminate at or proximate to a through-hole 4331 to provide the gas combination to the reservoir 4216.

In one embodiment, the microfluidic structure 4330 includes a plurality of first microfluidic channels configured to couple the gas inlets 4412 to each of the plurality of reservoirs 4216. In some embodiments, a first subset of the plurality of first microfluidic channels is configured to convey one or more of gaseous oxygen, nitrogen, or carbon dioxide to the reservoirs 4216, and a second subset of the plurality of first microfluidic channels is configured to convey liquid reagents to the reservoirs 4216. In a further embodiment, the microfluidic structure 4330 includes a plurality of second microfluidic channels configured to convey a gas away from the reservoirs 4216. In yet a further embodiment, the microfluidic structure 4330 comprises an injection molded plastic that is glued to the sample container 4214 for single-use application. The microfluidic structure 4330, sometimes referred to as a gassing chip, may comprise a body that is planar with flat top and bottom surfaces.

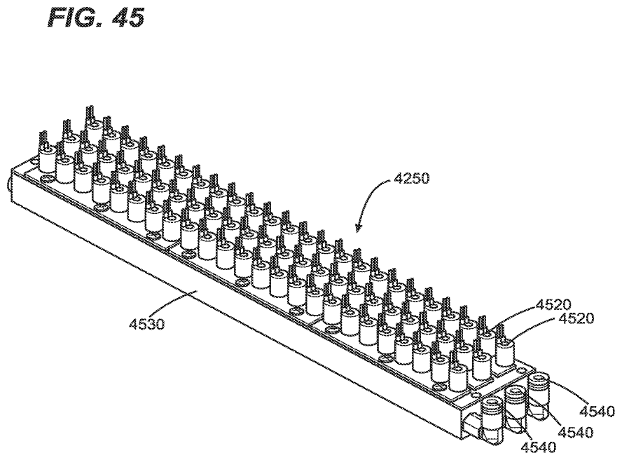

FIG. 45 is a perspective view of the valve array 4250. The valve array 4250 includes a grid or rows of valves 4520 situated on a valve base 4530. Each valve 4520 is configured to control a precise amount of gas to be delivered through a corresponding gas tube 4230 coupled with the valve 4520. The valve array 4250 includes one or more gas ports 4540 each configured to receive gas from one or more gas sources (not shown). As mentioned previously, the fluid at the sources may be in gaseous or liquid form. In continuing with the example above, the valve array 4250 may include three gas ports 4540, one for each of nitrogen, oxygen, and carbon dioxide. And, each gas port 4540 may provide a supply of gas to a row of twenty-four valves 4520. The valves 4520 of the valve array 4250 (e.g., seventy-two total) may correspond and fluidly couple with the gas tubes 4230 and microfluidic channels 4410 of the microfluidic structure 4330. Thus, in this example, each column or subgroup of valves 4520 (e.g., column of three valves 4520) may control a precise amount of nitrogen, oxygen, and carbon dioxide for each of the twenty-four reservoirs 4216.

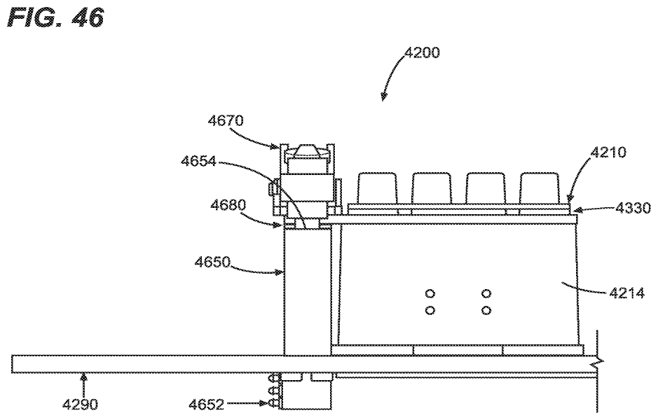

FIG. 46 is a side view of the bioreactor system 4200. As shown here, the bioreactor system 4200 may include a connector element 4650 with tube ports 4652 configured to receive or couple with gas tubes 4230. The connector element 4650 includes a structural housing with internal passages (see FIG. 42) for fluidly coupling the gas tubes 4230 with the gas inlets 4412 of the microfluidic structure 4330. A bottom portion of the connector element 4650 may be attached or coupled with the shaking table 4290, and the tube ports 4652 may be disposed below the shaking table 4290. A top portion or top end of the connector element 4650 may be coupled with the microfluidic structure 4330 to provide the gases to the gas inlets 4412. That is, the sample container 4214 and connector element 4650 may have a corresponding height such that, when situated adjacent to one another on the shaking table 4290, a portion of the microfluidic structure 4330 having the gas inlets 4412 is disposed over a top surface 4654 of the connector element 4650.

The bioreactor system 4200 also includes one or more fixture elements 4670 to secure and/or seal the sample container assembly 4210. In some embodiments, a seal 4680 or elastic member is provided between the connector element 4650 and the microfluidic structure 4330. Alternatively or additionally, the seal 4680 is provided between the microfluidic structure 4330 and the sample container 4214. The fixture element 4670 provides a clamping mechanism to compress the seal 4680 for airtight connections between components of the sample container assembly 4210 and/or connector element 6450.

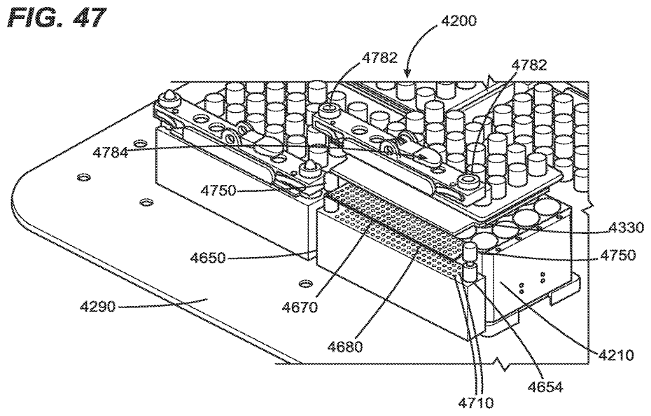

FIG. 47 is a perspective view of the bioreactor system 4200. In particular, FIG. 47 shows an example of a bioreactor system 4200 that includes four sample container assemblies 4210 coupled to the shaking table 4290 for cell culture experimentation (two sample container assemblies are visible in the foreground and portions of two other sample container assemblies are visible in the background). The sample container assembly on the left foreground shows an assembly that has been clamped in place, and the sample container assembly on the right foreground shows an exploded view of an assembly (purely for illustrative purposes). Each sample container assembly 4210 couples with a respective connector element 4650 attached to the shaking table 4290. The connector element 4650 includes one or more posts 4750 extending upward from the top surface 4654 of the connector element 4650. The posts 4750 couple with one or more post guides 4782 of the connector element 4650.

The top surface 4654 of the connector element 4650 also includes gas pathways 4710 configured to align and fluidly couple with the gas inlets 4412 of the microfluidic structure 4330. The seal 4680, being situated between the top surface 4654 of the connector element 4650 and a bottom surface of the microfluidic structure 4330, may include corresponding aligned gas pathways. After aligning the microfluidic structure 4330 with respect to the connector element 4650, the fixture element 4670 installs over the microfluidic structure 4330 to mate the post guides 4782 with the posts 4750. A lever 4784 of the fixture element 4670 is actuated to clamp the fixture element 4670 to the connector element 4650, thus providing pressure that seals the gas pathways to the microfluidic structure 4330.

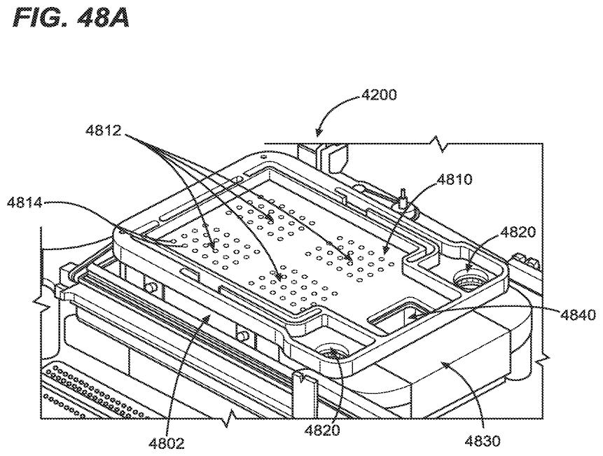

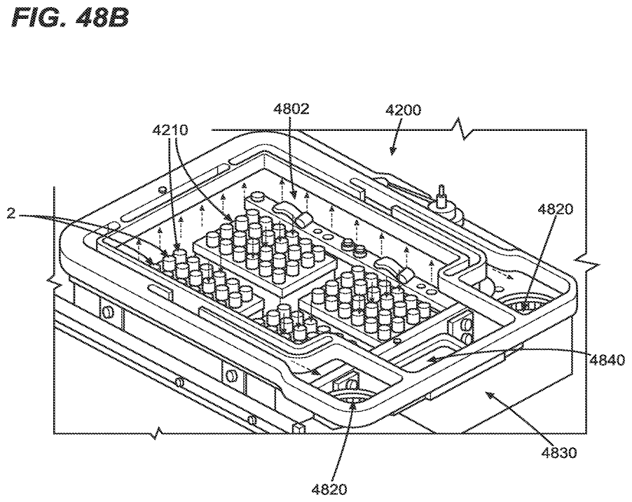

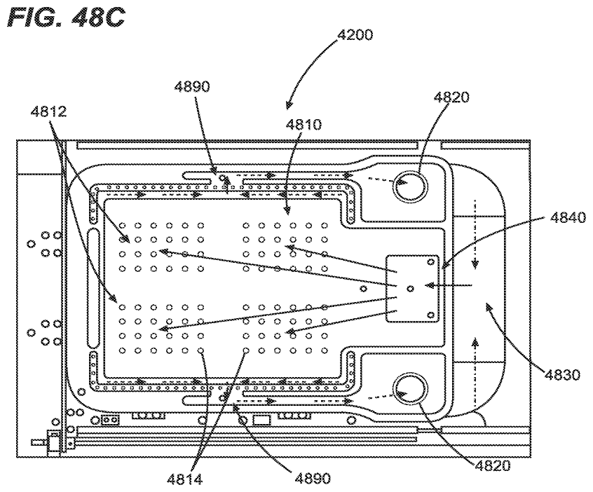

It may be important to have precise temperature control to allow for efficient cell cultivation. This may be especially true in the case of mammalian cells. As described in greater detail below, the bioreactor system 4200 may include one or more chambers above and/or below the sample container assemblies 4210, and temperature within these chambers may be controlled so as to regulate temperature within the sample container assemblies. FIG. 48A is a perspective view of components related to an upper chamber 4802 of the bioreactor system 4200. FIG. 48B is another perspective view of components related to the upper chamber 4802 of the bioreactor system 4200. FIG. 48C is a top view of components related to the upper chamber 4802 of the bioreactor system 4200.

As shown in FIG. 48A and FIG. 48C, the bioreactor system 4200 may include a cover inlay 4810 disposed over the sample container assemblies 4210 (cover inlay 4810 not shown in FIG. 48B). The bioreactor system 4200 includes one or more fans 4820 and a temperature control module 4830. As described in greater detail below, the fans and temperature control module 4830 may be used to create an air flow that moves heated air around the upper chamber and ultimately over the sample container assemblies 4210 so as to uniformly and precisely distribute heat among the sample container assemblies 4210.

In the illustrated example, the cover inlay 4810 forms a lid of the upper chamber 4802 or environment in which the sample container assemblies 4210 are placed and shaken. The cover inlay 4810 includes one or more inflow arrays 4812 or grid of vent holes 4814 that correspond or align over respective reservoirs 4216 of the sample container assemblies 4210. For example, for a format of four sample container assemblies 4210 each having twenty-four reservoirs 4216, the cover inlay 4810 may include four inflow arrays 4812 each having twenty-four vent holes 4814.

The fans 4820 are configured to draw air from the upper chamber 4802 and the push the air toward the temperature control module 4830. The fans 4820 may comprise radial fans disposed at edge or corner sidewalls of the upper chamber 4802. The temperature control module 4830 is configured to warm or cool the air by changing its surface temperature to maintain a target air temperature. For example, the temperature control module 4830 may comprise a Peltier module coupled with a heat sink and may include or connect with one or more temperature sensors to measure temperature of the upper chamber 4802, one or more optical sensors to measure temperature of the cultures, and/or a temperature controller for adjusting power of the Peltier module based on temperature measurement. The temperature control module 4830 may disposed at one end or side of the upper chamber 4802 between the fans 4820.

After air is tempered via the temperature control module 4830 it is pushed up through the tempered air passage 4840 and over the cover inlay 4810 (see e.g., arrows pointing toward vent holes 4814 in FIG. 48C). This tempered air is then pushed downward through each of the vent holes 4814 in the inflow arrays 4812, which channels the tempered air above each of the reservoirs of the sample container assemblies 4210 (see e.g., arrows pointing downward toward guide elements 2 in FIG. 48B). The inflow array 4812 of vent holes 4814 ensures that a directed airflow impinges on the desired position of the sample container assemblies 4210 (e.g., above each reservoir). This described configuration is advantageous over an alternative configuration that does not employ such a directed airflow in that the described configuration can provide uniform heating to each reservoir.

Once the air passes over the sample container assemblies, it may then be directed away from the sample container assemblies. In some embodiments, as illustrated in FIG. 48C, the air may be recirculated back to the temperature control module 4830 via outflows 4890 along the side walls of the bioreactor system 4200. In this example, the fans 4820 are configured to create a negative pressure by drawing in the air via these pathways and passing them over the temperature control module 4830 again so that the air temperature can be tempered again (e.g., heated/cooled to a desired temperature). Continued negative pressure from the fans 4820 may then cause this tempered air to again flow over the cover inlay 4810 and once again be pushed downward through the vent holes 4814 so as to heat/cool the sample container assemblies 4210.

In some embodiments, the bioreactor system 4200 may maintain a target temperature of the upper chamber 4802 and/or temperature inside the cultivation wells within one degree or within a half degree Celsius.

In some embodiments, diameters of the vent holes 4814 are varied according to a desired temperature distribution of the upper chamber 4802. For example, temperature inhomogeneity normally caused by the air traveling distances of variable length to different parts of the upper chamber 4802 may be counterbalanced by varying the diameters of the vent holes 4814. This allows different areas of the upper chamber 4802 to be supplied with streams of tempered air with different intensity. Additionally, although not shown in FIGS. 48A-C, it will be appreciated that a cover or sliding door disposed over the cover inlay 4810 may be provided to enclose the bioreactor system 4200.

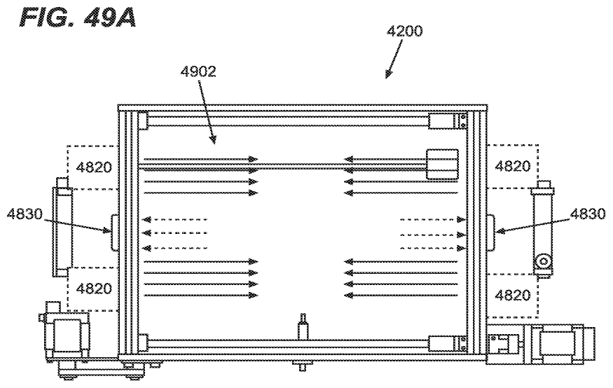

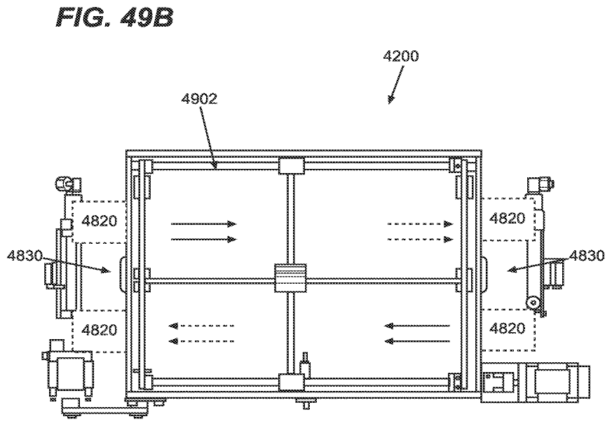

In some embodiments, the bioreactor system 4200 may alternatively or additionally include a lower chamber. FIG. 49A is a bottom view of components related to a lower chamber 4902 of the bioreactor system 4200, which is a chamber beneath the sample container assemblies 4210. FIG. 49B is another bottom view of components related to a lower chamber of the bioreactor system. In addition or alternative to tempering the upper chamber 4802, the bioreactor system 4200 may be configured to temper the lower chamber 4902 or environment below the shaking table 4290. For example, the lower chamber 4902 may be disposed directly underneath the shaking table 4290 and the sample container assemblies 4210 may stand on top of the shaking table 4290. In this example, two temperature control modules 4830 are provided at opposite ends or side walls of the lower chamber 4902. Additionally, each side wall includes fans 4820 on either side of the temperature control module 4830.

In the example shown in FIG. 49A, the fans 4820 are configured to draw air from a middle portion of the lower chamber 4902, to pass the air across or through the temperature control module 4830, and to blow the tempered air back into the lower chamber 4902 along its lateral sides. This may be performed synchronously on both opposite side walls of the lower chamber, as shown by the arrows in FIG. 49A. In the example shown in FIG. 49B, the fans 4820 are configured to cause the air to flow or circulate around the lower chamber 4902. For example, as heated air traverses the lower chamber 4902 and loses heat along the way, the cooled air is pulled in, passed over the temperature control module 4830 for reheating, and recirculated (e.g., clockwise direction shown in FIG. 49B) in the lower chamber 4902. The temperature control modules 4830 and fans 4820 may similarly cool the lower chamber 4902. Advantageously, the conditioned airflow may heat or cool a lower side or area underneath the sample container assemblies 4210.

In one embodiment, the bioreactor system 4200 is configured to temper both the upper chamber 4802 and the lower chamber 4902. Advantageously, an evaporation rate of the bioreactor system 4200 is controlled by separately adjusting the temperature of the upper chamber 4802 and the lower chamber 4902. For example, to achieve a target cultivation temperature (e.g., approximately thirty-seven degrees Celsius), the upper chamber 4802 may be set to a first temperature (e.g., approximately thirty-nine degrees Celsius) slightly higher than the target temperature, and lower chamber 4902 may be set to a second temperature (e.g., approximately thirty-six degrees Celsius) slightly lower than the target temperature to prevent condensation due to water contained within components of the bioreactor system 4200.

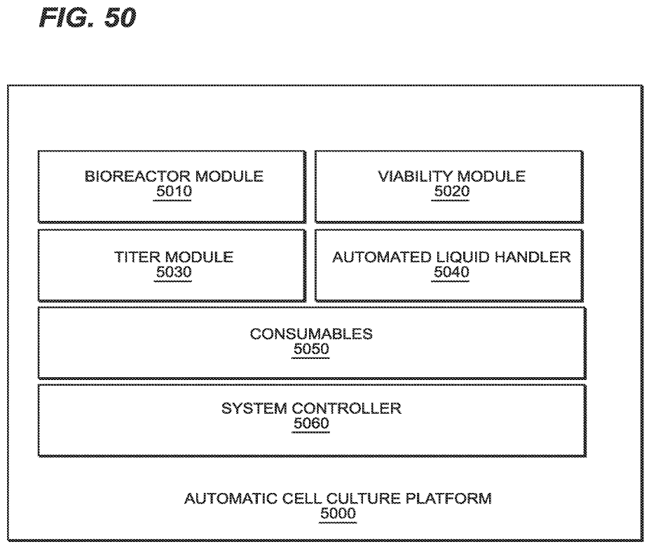

FIG. 50 is a block diagram of an automatic cell culture platform 5000. The automatic cell culture platform 5000 includes a bioreactor module 5010 (e.g., bioreactor system 4200), a viability module 5020, a titer module 5030, an automated liquid handler 5040, consumables 5050, and a system controller 5060. The automatic cell culture platform 5000 thus includes integrated cell health, titer, and cell media measurement capabilities. Additionally, the automatic cell culture platform 5000 is configured to miniaturize design of experiment in mammalian cell line development and process development to accelerate experimentation and reduce hands-on time.

As previously described, the bioreactor module 5010 (e.g., bioreactor system 4200) may include at least reaction reservoirs (e.g., 5 mL reservoirs) to support an increased number of samples in a single system. For example, four disposable microtiter plates with twenty-four 5 mL wells each are housed on a shaking platform (e.g., 3 mm diameter circular orbit at 200-800 RPM). Each well may include integrated optodes to measure pH and DO2. The bioreactor module 5010 may also include an optical measurement system to measure each well. For example, the bioreactor module 5010 may include or connect with a control system for a sample container assembly 4210 with a gassing lid (e.g., microfluidic structure 4330). The control system may include sensors configured to acquire measurement parameters associated with the sample container assembly 4210, a gas supply system providing at least one gas to the gassing lid, and a controller configured to process the acquired measurement parameters and control the gas supply based upon the processed measurement parameters.

The viability module 5020 may be configured to provide measurement of cell concentration and viability for a specimen taken from the wells. For example, the automated liquid handler 5040 may aspirate a sample from a reservoir of the bioreactor module 5010 and introduce it to the viability module 5020, which may measure a protein concentration of the sample. As another example, the sample may be aspirated from a reagent tube or a reagent well from a deck of the automatic cell culture platform 5000 (i.e., the sample need not be from the bioreactor module 5010). The titer module 5030 may be configured to measure protein concentrations (e.g., Immunoglobulin G (IgG) concentration). For example, the automated liquid handler 5040 may aspirate a sample from a reservoir of the bioreactor module 5010 and introduce it to the titer module 5030, which may measure a protein concentration of the sample. As another example, the sample may be aspirated from a reagent tube or a reagent well from a deck of the automatic cell culture platform 5000 (i.e., the sample need not be from the bioreactor module 5010). Any suitable titer measurement method may be employed (e.g., florescence polarization measurements of the sample). The automated liquid handler 5040 may include one or more fixed probes and/or one or more disposable probes for sampling a cell cultivation reservoir (e.g., of the sample container assemblies 4210) or feeding/adding reagents to the cell cultivation reservoir. The consumables 5050 may include one or more of: gas supply (e.g., nitrogen, oxygen, and carbon dioxide), bioreactor microtiter plates (e.g., sample container assemblies 4210) that are disposable to support individual gassing, reagents for the titer module 5030, reagents for the viability module 5020, reagents for tip cleaning, and/or other cell growth reagents (e.g., customer-supplied cell growth media).

The system controller 5060 is operatively coupled to control the operations of the automatic cell culture platform 5000. The system controller 5060 may therefore be operatively coupled to the bioreactor module 5010, viability module 5020, titer module 5030, and/or automated liquid handler 5040, as well as respective underlying components, for carrying out the functionalities described herein. The system controller 5060 may also comprise a computer storage medium that stores, in a tangible and non-transitory manner, a computer program product, that when executed by the system controller 5060, causes a processor of the system controller 5060 to carry out the functionalities described herein. Additionally, the system controller 5060 may include computer interaction elements (e.g., keyboard, mouse, touch screen, graphical user interface, etc.) for receiving user input.

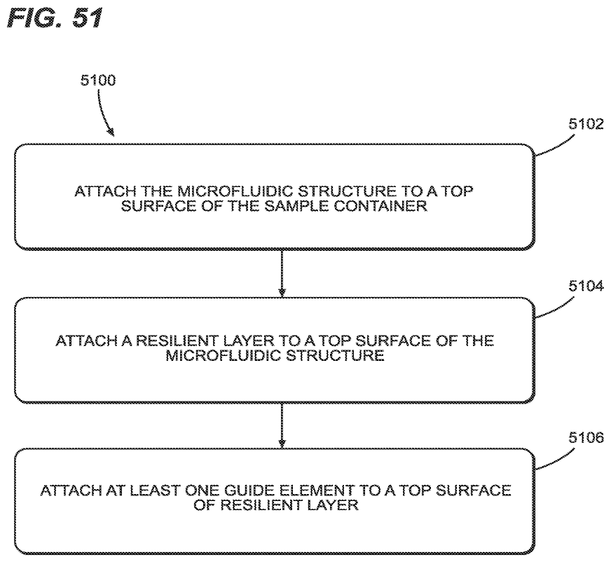

FIG. 51 illustrates an example of a method 5100 of assembling the sample container assembly 4210. The method 5100 can include an operation 5102 of attaching the microfluidic structure 4330 to a top surface of the sample container 4214. Operation 5102 can include adhering the microfluidic structure 4330 to the top surface of the sample container 4214. Next, the method 5100 can include an operation 5104 of attaching a resilient layer (e.g., resilient layer 4310) to a top surface of the microfluidic structure 4330. Next, the method 5100 can include an operation 5106 of attaching at least one guide element 2 to a top surface of the resilient layer.

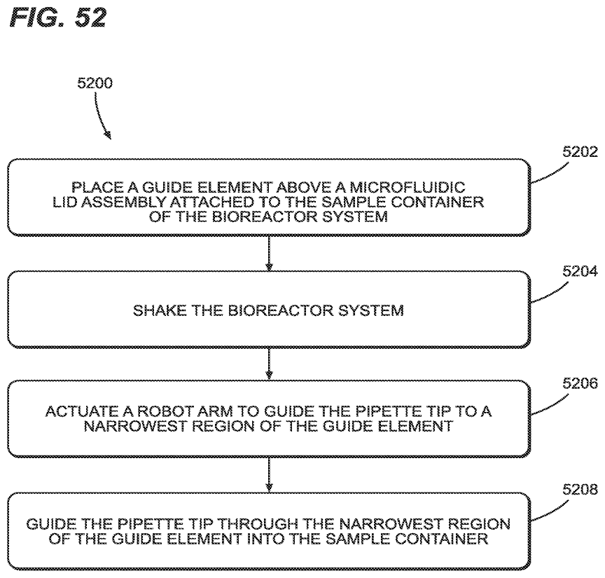

FIG. 52 illustrates an example of a method 5200 of inserting a pipette tip into sample container while a bioreactor system is being shaken. The method 5200 can include an operation 5202 of placing the guide element 2 above the microfluidic lid assembly 4212 attached to the sample container 4214 of the bioreactor system 4200. Next, the method 5200 can include an operation 5204 of shaking the bioreactor system 4200. For example, operation 5204 can include operating the shaking table 4290 to shake the sample container assembly 4210 by moving the sample container assembly 4210 within a predetermined range of motion. In one embodiment, the predetermined range of motion is within one or more interior diameters of one or more top ends of one or more of the guide elements 2.

Next, the method 5200 can include an operation 5206 of actuating a robot arm (e.g., of pipetting robot 70) to guide the pipette tip 71 to a narrowest region of the guide element 2. Next, the method 5200 can include an operation 5208 of guiding the pipette tip 71 through the narrowest region of the guide element 2 into the sample container 4214. Thus, in the method 5200, an automated pipettor comprising one or more pipettors is configured to insert one or more pipette tips 71 into the sample container 4214 via the one or more guide elements 2 while the sample container assembly 4210 is being shaken.

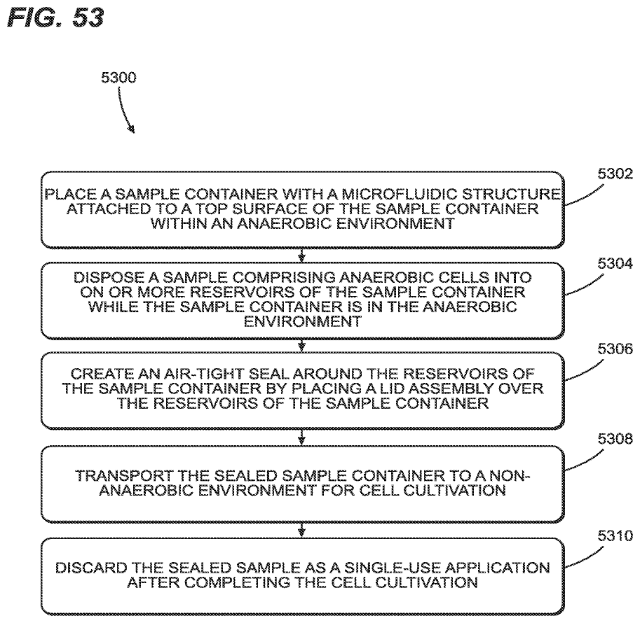

FIG. 53 illustrates an example of a method 5300 of cultivating anaerobic cells. The method 5300 can include an operation 5302 of placing the sample container 4214 with the microfluidic structure 4330 attached to a top surface of the sample container 4214 within an anaerobic environment. Next, the method 5300 can include an operation 5304 of disposing a sample comprising anaerobic cells into one or more reservoirs 4216 of the sample container 4214 while the sample container 4214 is in the anaerobic environment. Since the guide structure 1 and guide elements 2 may not yet be attached, the operation 5304 may be performed with a large pipette.

Next, the method 5300 can include an operation 5306 of creating an air-tight seal around the reservoirs 4216 of the sample container 4214 by placing a lid assembly (e.g., components of microfluidic lid assembly 4212) over the reservoirs 4216 of the sample container. Next, the method 5300 can include an operation 5308 of transporting the sealed sample container to a non-anaerobic environment for cell cultivation. Next, the method 5300 can include an operation 5310 of discarding the sealed sample as a single-use application after completing the cell cultivation.

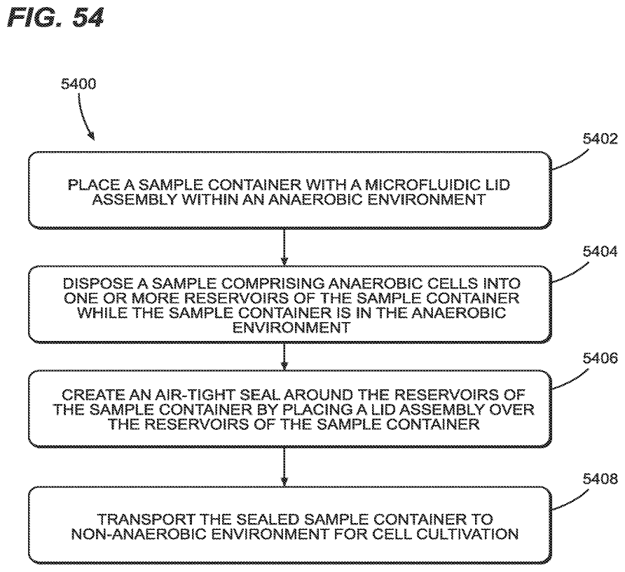

FIG. 54 illustrates another example of a method 5400 of cultivating anaerobic cells. The method 5400 can include an operation 5402 of placing the sample container 4214 with a microfluidic lid assembly 4212 within an anaerobic environment. Next, the method 5400 can include an operation 5404 of disposing a sample comprising anaerobic cells into one or more reservoirs 4216 of the sample container 4214 while the sample container 4214 is in the anaerobic environment.

Next, the method 5400 can include an operation 5406 of creating an air-tight seal around the reservoirs of the sample container by placing a lid assembly over the reservoirs of the sample container. Next, the method 5400 can include an operation 5408 of transporting the sealed sample container to a non-anaerobic environment for cell cultivation.

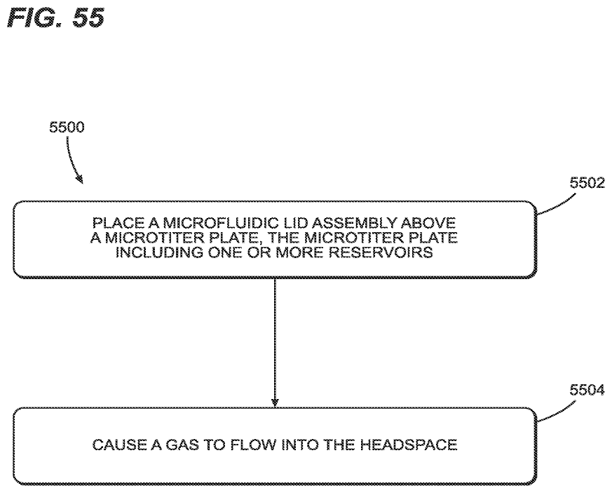

FIG. 55 illustrates an example of a method 5500 of controlling gas concentrations in a headspace above reservoirs of a microtiter plate. The method 5500 can include an operation 5502 of placing a microfluidic lid assembly 4214 above the microtiter plate (e.g., sample container 4214), the microtiter plate including one or more reservoirs 4216. In one embodiment, the microfluidic lid assembly 4214 is configured to provide a headspace above the reservoirs 4216 to allow gas exchange during cell cultivation. In a further embodiment, the headspace above the reservoirs is 20 mL to 400 mL. Next, the method 5500 can include an operation 5404 of causing a gas to flow into the headspace.

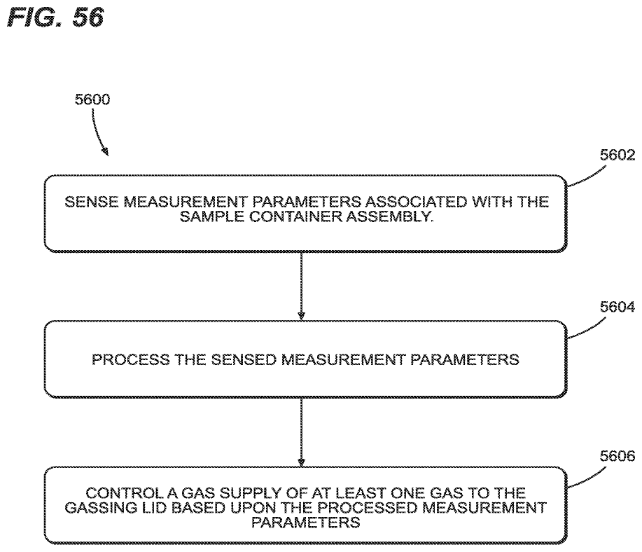

FIG. 56 illustrates an example of a method 5600 of controlling a sample container assembly with a gassing lid (e.g., microfluidic structure 4330). The method 5600 can include an operation 5602 of sensing measurement parameters associated with the sample container assembly 4210. Next, the method 5600 can include an operation 5604 of processing the sensed measurement parameters. Next, the method 5600 can include an operation 5606 of controlling a gas supply of at least one gas to the gassing lid based upon the processed measurement parameters.

Additional aspects of the present disclosure are listed in the following clauses.

Clause 1. A lid assembly comprising: a lid housing having a top exterior surface and a bottom interior surface, the lid housing configured to cover a sample container; a first resilient layer disposed in the lid housing; and a sealing surface projecting from the bottom interior surface of the lid housing toward the first resilient layer to create an air-tight seal when the sealing surface is pressed against the first resilient layer.

Clause 2. The lid assembly of claim 1, the first resilient layer including one or more first apertures aligned with a respective guide element, each first aperture being configured to open when a pipette tip is pushed through and to close when the pipette tip is removed.

Clause 3. A lid assembly comprising: a lid housing having a top exterior surface and a bottom interior surface, the lid housing configured to cover a sample container; one or more guide elements extending from the top exterior surface of the lid housing, each guide element having a hollow interior portion running from a top end to a bottom end, the hollow interior portion having a larger cross-sectional area at the top end than at the bottom end, and each guide element being configured to receive and guide a pipette tip; and a first layer disposed in the lid housing, the first layer including one or more first apertures aligned with a respective guide element, each first aperture being configured to open when the pipette tip is pushed through and to close when the pipette tip is removed.

Clause 4. The lid assembly of claim 3, further comprising: a sealing surface projecting from the bottom interior surface of the lid housing toward the first layer to create an air-tight seal when the sealing surface is pressed against the first layer.

Clause 5. The lid assembly of claim 4, wherein the sealing surface includes a partition dividing a first recessed area on the bottom interior surface of the lid housing from a second recessed area on the bottom interior surface of the lid housing.

Clause 6. The lid assembly of claim 5, wherein the sealing surface and the partition are continuous with one another.

Clause 7. The lid assembly of claim 5, further comprising a first gas port connected to the first recessed area of the lid housing and configured to receive a pressurizing gas.

Clause 8. The lid assembly of claim 7, further comprising second and third gas ports configured to receive or remove one or more gases from the second recessed area.

Clause 9. The lid assembly of claim 5, further comprising: one or more additional partitions configured to separate additional recessed areas between the bottom interior surface of the lid housing and the first layer.

Clause 10. The lid assembly of claim 4, wherein the sealing surface is made of a rigid material.

Clause 11. The lid assembly of claim 4, wherein the sealing surface is made of PEEK.

Clause 12. The lid assembly of claim 3, further comprising: a sterile layer disposed on a bottom side of the first layer, wherein the sterile layer is configured to be pierced by the pipette tip.

Clause 13. The lid assembly of claim 3, further comprising: a second layer disposed between the bottom end of each guide element and the top exterior surface of the lid housing, the second layer having one or more second apertures aligned with respective guide elements and first apertures, and providing access to one or more through-holes in the lid housing, each second aperture being configured to open when the pipette tip is pushed through the second aperture and to close when the pipette tip is removed.

Clause 14. The lid assembly of claim 3, further comprising one or more posts extending from the bottom interior surface of the lid housing toward the first layer.

Clause 15. The lid assembly of claim 3, wherein the one or more guide elements form an integral part of the lid housing.

Clause 16. The lid assembly of claim 15, wherein the one or more guide elements are removably coupled to the top exterior surface of the lid housing.

Clause 17. The lid assembly of claim 15, wherein the hollow interior portions has a conical or frustoconical shape.

Clause 18. The lid assembly of claim 15, wherein the one or more first apertures are slits.

Clause 19. The lid assembly of claim 18, wherein the slits are self-healing.

Clause 20. The lid assembly of claim 3, wherein the first layer is a resilient polymer material.

Clause 21. The lid assembly of claim 3, wherein the first layer is made from silicone.

Clause 22. A container assembly comprising: a lid assembly comprising; a lid housing with a top exterior surface and a bottom interior surface, the lid housing configured to cover a sample container; one or more guide elements extending from the top exterior surface of the lid housing, each guide element having a hollow interior portion running from a top end to a bottom end, the hollow interior portion having a larger cross-sectional area at the top end than at the bottom end, and each guide element being configured to receive and guide a pipette tip; and a first layer disposed in the lid housing, the first layer having one or more first apertures aligned with respective guide elements, each first aperture being configured to open when the pipette tip is pushed through and to close when the pipette tip is removed; and a sample container comprising a plurality of wells.

Clause 23. The container assembly of claim 22, wherein a first portion of the sample container comprises one or more first wells and a second portion of the sample container comprises one or more second wells, wherein the one or more first wells are configured to contain fluid reagents and the one or more second wells are configured to contain a fluid sample comprising one or more cells, wherein one or more of the first wells are fluidically coupled to one or more of the second wells via one or more fluidic channels; wherein the lid assembly provides an air-tight seal around the sample container when the lid assembly is caused to be compressed against the sample container.

Clause 24. The container assembly of claim 23, wherein the air-tight seal has a first sealing surface on the sample container and a second sealing surface on the lid assembly pressing against the first sealing surface whereby both sealing surfaces act perpendicular to the bottom interior surface of the lid housing.

Clause 25. The container assembly of claim 23, further comprising: an eccentric lever and a ball sleeve comprising radially guided balls.

Clause 26. A bioreactor system comprising: a reversibly sealable sample container assembly comprising: a lid assembly comprising: a lid housing having a top exterior surface and a bottom interior surface, the lid housing configured to cover a sample container; one or more guide elements extending from the top exterior surface of the lid housing, each guide element having a hollow interior portion running from a top end to a bottom end, the hollow interior portion having a larger cross-sectional area at the top end than at the bottom end, and each guide element being configured to receive and guide a pipette tip; a first layer disposed in the lid housing, the first layer having one or more first apertures aligned with respective guide elements, each first aperture being configured to open when the pipette tip is pushed through and to close when the pipette tip is removed; a sample container comprising a plurality of wells; a platform configured to shake the sample container assembly by moving the sample container assembly within a predetermined range of motion, wherein the predetermined range of motion is within one or more interior diameters of one or more top ends of one or more of the guide elements; and pipetting robot having one or more pipette tips configured for insertion into the sample container via the one or more guide elements while the sample container assembly is being shaken.

Clause 27. The bioreactor system of claim 26, wherein the platform is configured to move the sample container assembly in an orbital fashion.

Clause 28. The bioreactor system of claim 26, wherein the platform is configured to move the sample container assembly in an orbital fashion within a range of 600 RPM to 1000 RPM.

Clause 29. The bioreactor system of claim 26, wherein the platform is configured to move the sample container assembly in an orbital fashion within a range of 600 RPM to 800 RPM.

Clause 30. The bioreactor system of claim 26, 28, or 29, wherein an agitation diameter of the orbital movement of the sample container assembly is within a range of 1 mm to 5 mm.

Clause 31. A method of sealing a sample container comprising: placing a sterile layer on top of the sample container; placing a resilient layer on top of the sterile layer; pressing a lid housing on top of the resilient layer; and releasably securing the lid housing to the sample container.

Clause 32. The method of claim 31, further comprising actuating an eccentric lever and ball sleeve comprising radially guided balls.

Clause 33. The method of claim 31, further comprising actuating release pins to release the sample container from the lid housing.

Clause 34. A method of cultivating anaerobic cells, the method comprising: placing a sample container within an anaerobic environment; disposing a sample comprising anaerobic cells into one or more wells of the sample container while the sample container is in the anaerobic environment; creating an air-tight seal around the wells of the sample container by placing a lid assembly over the wells of the sample container; and transporting the sealed sample container to a non-anaerobic environment for cell cultivation.

Clause 35. The method of claim 34, wherein the sealed sample container is placed within a microbioreactor disposed in the non-anaerobic environment.

Clause 36. The method of claim 34, wherein the sample container and lid assembly define a headspace above the one or more wells, and the method further comprises: adjusting an oxygen concentration in the headspace to between 0% and 5%.

Clause 37. The method of claim 34, wherein the sample container and lid assembly define a headspace above the one or more wells, and the method further comprises: adjusting an oxygen concentration in the headspace to between 0% and 10%.

Clause 38. The method of claim 34, wherein the sample container and lid assembly define a headspace above the one or more wells, and the method further comprises: adjusting an oxygen concentration in the headspace to between 0% and 20%.

Clause 39. A method of inserting a pipette tip into sample container while a bioreactor system is being shaken, the method comprising: placing a guide element above the sample container of the bioreactor system; shaking the bioreactor system; actuating a pipetting robot to guide the pipette tip to a narrowest region of the guide element; and guiding the pipette tip through the narrowest region of the guide element into the sample container.

Clause 40. The method of claim 39, further comprising removing a volume of fluid from the sample container via the pipette tip.

Clause 41. The method of claim 39, further comprising adding a volume of fluid to the sample container via the pipette tip.

Clause 42. A lid assembly for a microplate, wherein the microplate includes one or more wells, the lid assembly being configured to provide a headspace above the wells to allow gas exchange during cell cultivation, wherein the headspace above the wells is 20 mL to 400 ml.

Clause 43. The lid assembly of claim 42, wherein the headspace is 60 ml to 90 ml.

Clause 44. A method of controlling gas concentrations in a headspace above wells of a microplate, the method comprising: placing a lid assembly above the microplate, the microplate including one or more wells, the lid assembly configured to provide a headspace above the wells to allow gas exchange during cell cultivation, wherein the headspace above the reservoirs is 20 mL to 400 mL; and causing a gas to flow into the headspace.

Clause 45. The method of claim 44, further comprising: measuring a concentration of the gas; and adjusting the gas flow based on the measured concentration.

Clause 46. A control system for a sample container assembly with a gassing lid, the control system comprising: sensors configured to acquire measurement parameters associated with the sample container assembly; a gas supply system providing at least one gas to the gassing lid; and a controller configured to process the acquired measurement parameters and control the gas supply based upon the processed measurement parameters.

Clause 47. A method of controlling a sample container assembly with a gassing lid, the method comprising: sensing measurement parameters associated with the sample container assembly; processing the sensed measurement parameters; and controlling a gas supply of at least one gas to the gassing lid based upon the processed measurement parameters.

Clause 48. A computer program product, that stores in a tangible and non-transitory manner, a computer program code, that when executed by a computer controller, causes the computer controller to: sense measurement parameters associated with a sample container assembly having a gassing lid; process the sensed measurement parameters; and control a gas supply of at least one gas to the gassing lid based upon the processed measurement parameters.

Clause 49. A microfluidic lid assembly for creating an air-tight seal above a sample container, the lid assembly comprising: a microfluidic structure configured to be disposed over a plurality of reservoirs of the sample container to create a seal along an outside perimeter of the sample container, wherein the microfluidic structure comprises: one or more gas inlets for receiving one or more connections to one or more fluid sources; and a plurality of first microfluidic channels configured to couple the gas inlets to each of the plurality of reservoirs; wherein the microfluidic structure separates each of the reservoirs from a plurality of guide elements and a layer with apertures disposed over the reservoirs of the sample container.

Clause 50. The microfluidic lid assembly of claim 49, wherein the microfluidic structure is configured to individually seal each of the plurality of reservoirs.

Clause 51. The microfluidic lid assembly of claim 50, wherein each of the plurality of first microfluidic channels is configured transport a controlled gas concentration to an individually sealed one of the plurality of reservoirs.

Clause 52. The microfluidic lid assembly of claim 49, wherein at least a first subset of the plurality of first microfluidic channels is configured to convey one or more of gaseous oxygen, nitrogen, or carbon dioxide to the reservoirs.

Clause 53. The microfluidic lid assembly of claim 52, wherein a second subset of the plurality of first microfluidic channels is configured to convey liquid reagents to the reservoirs.

Clause 54. The microfluidic lid assembly of claim 49, further comprising a plurality of second microfluidic channels configured to convey a gas away from the reservoirs.

Clause 55. The microfluidic lid assembly of claim 49, wherein the plurality of guide elements and the layer form an integral unit.

Clause 56. The microfluidic lid assembly of claim 49, wherein the plurality of guide elements are disposed on a guide structure that is coupled to the layer.

Clause 57. The microfluidic lid assembly of claim 49, wherein the microfluidic lid assembly is configured to be adhered to the sample container with an adhesive.

Clause 58. The microfluidic lid assembly of claim 49, wherein the apertures comprise slits in the layer.

Clause 59. The microfluidic lid assembly of claim 49, wherein the layer comprises a resilient polymer material.

Clause 60. A sample container assembly comprising: a sample container comprising a plurality of reservoirs; and a microfluidic structure comprising: one or more gas inlets and a plurality of microfluidic channels; and wherein a bottom surface of the microfluidic structure is adhered to a top surface of the sample container.

Clause 61. The sample container assembly of claim 60, wherein the plurality of guide elements are disposed on a guide structure that is adhered to a top surface of the layer, and wherein a top surface of the microfluidic structure is adhered to a bottom surface of the layer.

Clause 62. A bioreactor system comprising: a sample container assembly comprising: a sample container comprising a plurality of reservoirs; a microfluidic structure comprising one or more gas inlets and a plurality of microfluidic channels, wherein a bottom surface of the microfluidic structure is adhered to a top surface of the sample container; one or more guide elements positioned above the microfluidic structure; a shaking table configured to shake the sample container assembly by moving the sample container assembly within a predetermined range of motion, wherein the predetermined range of motion is within one or more interior diameters of one or more top ends of one or more of the guide elements; and an automated pipettor comprising one or more pipettors configured to insert one or more pipette tips into the sample container via the one or more guide elements while the sample container assembly is being shaken.

Clause 63. The bioreactor system of claim 62 further comprising: an upper chamber disposed above the shaking table; and a cover inlay configured to direct tempered air in the upper chamber to uniformly temper each of the plurality of reservoirs.

Clause 64. The bioreactor system of claim 63 wherein the cover inlay includes vent holes that align with the plurality of reservoirs, the vent holes configured to direct the tempered air.

Clause 65. The bioreactor system of claim 62 further comprising: a lower chamber disposed below the shaking table; and one or more fans configured to circulate tempered air around the lower chamber.

Clause 66. The bioreactor system of claim 62 further comprising: an upper chamber disposed above the shaking table; a lower chamber disposed below the shaking table; one or more first temperature control modules configured to temper air of the upper chamber at a first target temperature; and one or more second temperature control modules configured to temper air of the lower chamber at a second target temperature.

Clause 67. The bioreactor system of claim 66 wherein: the first temperature is set higher than the second temperature to prevent condensation in the bioreactor system.

Clause 68. A method of assembling a sample container assembly comprising: attaching a microfluidic structure to a top surface of a sample container; attaching a resilient layer to a top surface of the microfluidic structure; and attaching at least one guide element to a top surface of the resilient layer.

Clause 69. The method of claim 68 further comprising adhering the microfluidic structure to the top surface of the sample container.

Clause 70. A method of inserting a pipette tip into sample container while a bioreactor system is being shaken, the method comprising: placing a guide element above a microfluidic lid assembly attached to the sample container of the bioreactor system; shaking the bioreactor system; actuating a robot arm to guide the pipette tip to a narrowest region of the guide element; and guiding the pipette tip through the narrowest region of the guide element into the sample container.

Clause 71. A method of cultivating anaerobic cells, the method comprising: placing a sample container with a microfluidic structure attached to a top surface of the sample container within an anaerobic environment; disposing a sample comprising anaerobic cells into one or more reservoirs of the sample container while the sample container is in the anaerobic environment; creating an air-tight seal around the reservoirs of the sample container by placing a lid assembly over the reservoirs of the sample container; and transporting the sealed sample container to a non-anaerobic environment for cell cultivation.

Clause 72. A method of controlling gas concentrations in a headspace above reservoirs of a microtiter plate, the method comprising: placing a microfluidic lid assembly above the microtiter plate, the microtiter plate including one or more reservoirs, the microfluidic lid assembly configured to provide a headspace above the reservoirs to allow gas exchange during cell cultivation, wherein the headspace above the reservoirs is 20 mL to 400 mL; and causing a gas to flow into the headspace.

Clause 73. A method of cultivating anaerobic cells, the method comprising: placing a sample container with a microfluidic lid assembly within an anaerobic environment; disposing a sample comprising anaerobic cells into one or more reservoirs of the sample container while the sample container is in the anaerobic environment; creating an air-tight seal around the reservoirs of the sample container by placing a lid assembly over the reservoirs of the sample container; and transporting the sealed sample container to a non-anaerobic environment for cell cultivation.

Clause 74. A control system for a sample container assembly with a gassing lid, comprising: sensors configured to acquire measurement parameters associated with the sample container assembly; a gas supply system providing at least one gas to the gassing lid; and a controller configured to process the acquired measurement parameters and control the gas supply based upon the processed measurement parameters.

Clause 75. A method of controlling a sample container assembly with a gassing lid, comprising: sensing measurement parameters associated with the sample container assembly; processing the sensed measurement parameters; and controlling a gas supply of at least one gas to the gassing lid based upon the processed measurement parameters.

Clause 76. A computer program product, that stores in a tangible and non-transitory manner, a computer program code, that when executed by a computer controller, causes the computer controller to: sense measurement parameters associated with a sample container assembly having a gassing lid; process the sensed measurement parameters; and control a gas supply of at least one gas to the gassing lid based upon the processed measurement parameters

Clause 77. An automatic cell culture system, comprising: a titer module; and a bioreactor module including cell health and cell media measurement capabilities integrated with the titer module.

Clause A1. A system comprising: a microfluidic lid assembly configured to create an air-tight seal above a sample container having reservoirs, the microfluidic lid assembly comprising: guide elements; a layer with apertures configured to align underneath the guide elements; and a microfluidic structure with through-holes configured to align underneath the apertures of the layer, wherein the microfluidic structure comprises: gas inlets configured to fluidly couple with one or more fluid sources; and microfluidic channels configured to fluidly couple the gas inlets to the reservoirs of the sample container.

Clause A2. The system of claim A1, wherein the microfluidic structure is configured to individually seal each of the reservoirs of the sample container.

Clause A3. The system of claim A2, wherein each microfluidic channel is configured transport a controlled gas concentration to an individually sealed one of the plurality of reservoirs.

Clause A4. The system of claim A1, wherein a first subset of the microfluidic channels is configured to convey one or more of gaseous oxygen, nitrogen, or carbon dioxide to the reservoirs.

Clause A5. The system of claim A4, wherein a second subset of the microfluidic channels is configured to convey liquid reagents to the reservoirs.

Clause A6. The system of claim A1, wherein the microfluidic structure further comprises additional microfluidic channels configured to convey a gas away from the reservoirs.

Clause A7. The system of claim A1, wherein the guide elements and the layer form an integral unit.

Clause A8. The system of claim A1, wherein the guide elements are disposed on a guide structure that is coupled to the layer.

Clause A9. The system of claim A1, wherein the microfluidic lid assembly is configured to be adhered to the sample container with an adhesive.

Clause A10. The system of claim A1, wherein the apertures comprise slits in the layer.

Clause A11. The system of claim A1, wherein the layer comprises a resilient polymer material.

Clause A12. The system of claim A1, further comprising: a sample container assembly, comprising: the sample container comprising the reservoirs; and the microfluidic structure, wherein a bottom surface of the microfluidic structure is adhered to a top surface of the sample container.

Clause A13. The system of claim A12, wherein a top surface of the microfluidic structure is adhered to a bottom surface of the layer.

Clause A14. The system of claim A12, further comprising: a bioreactor system, comprising: the sample container assembly; a shaking table configured to shake the sample container assembly by moving the sample container assembly within a predetermined range of motion, wherein the predetermined range of motion is within an interior diameter of a top end of a guide element; and an automated pipettor comprising one or more pipettors configured to insert one or more pipette tips into the sample container via the guide element while the sample container assembly is being shaken.

Clause A15. The system of claim A14, wherein the bioreactor system further comprises: an upper chamber disposed above the shaking table; and a cover inlay configured to direct tempered air in the upper chamber to uniformly temper each of the reservoirs.

Clause A16. The system of claim A15, wherein the cover inlay includes vent holes that align with the reservoirs, the vent holes configured to direct the tempered air.

Clause A17. The system of claim 14, wherein the bioreactor system further comprises: a lower chamber disposed below the shaking table; and one or more fans configured to circulate tempered air around the lower chamber.

Clause A18. The system of claim A14, wherein the bioreactor system further comprises: an upper chamber disposed above the shaking table; a lower chamber disposed below the shaking table; one or more first temperature control modules configured to temper air of the upper chamber at a first target temperature; and one or more second temperature control modules configured to temper air of the lower chamber at a second target temperature.

Clause A19. The system of claim A18, wherein the first temperature is set higher than the second temperature to prevent condensation in the bioreactor system.

Clause A20. The system of claim A14, further comprising: an automatic cell culture system, comprising: a titer module; and the bioreactor system, wherein the bioreactor system includes cell health and cell media measurement capabilities integrated with the titer module.

Clause A21. The system of claim A12, further comprising: a control system, comprising: sensors configured to acquire measurement parameters associated with the sample container assembly; a gas supply system configured to provide at least one gas to the microfluid structure; and a controller configured to process the acquired measurement parameters and control the gas supply system based upon the processed measurement parameters.

Clause A22. A method comprising: attaching a microfluidic structure to a top surface of a sample container; attaching a resilient layer to a top surface of the microfluidic structure; and attaching at least one guide element to a top surface of the resilient layer.

Clause A23. The method of claim A22, further comprising: adhering the microfluidic structure to the top surface of the sample container.

Clause A24. The method of claim A22, further comprising: shaking the sample container; actuating a robot arm to guide a pipette tip to a narrowest region of the at least one guide element; and guiding the pipette tip through the narrowest region of the at least one guide element into the sample container.

Clause A25. The method of claim A22, further comprising: placing the sample container with the microfluidic structure attached to the top surface of the sample container within an anaerobic environment; disposing a sample comprising anaerobic cells into one or more reservoirs of the sample container while the sample container is in the anaerobic environment; creating an air-tight seal around the reservoirs of the sample container by placing a lid assembly over the reservoirs of the sample container; and transporting the sealed sample container to a non-anaerobic environment for cell cultivation.

Clause A26. The method of claim A22, further comprising: placing a microfluidic lid assembly above the sample container, the sample container including reservoirs, the microfluidic lid assembly including the microfluidic structure, the resilient layer, and the at least one guide element, the microfluidic lid assembly configured to provide a headspace above the reservoirs to allow gas exchange during cell cultivation, wherein the headspace above the reservoirs is 20 mL to 400 mL; and causing a gas to flow into the headspace.

Clause A27. The method of claim A26, further comprising: placing the sample container within an anaerobic environment; disposing a sample comprising anaerobic cells into one or more reservoirs of the sample container while the sample container is in the anaerobic environment; creating an air-tight seal around the reservoirs of the sample container by attaching the microfluidic lid assembly to the top surface of the sample container; and transporting the sealed sample container to a non-anaerobic environment for cell cultivation.

Clause A28. The method of claim A22, further comprising: sensing measurement parameters associated with a sample container assembly comprising the sample container and the microfluidic structure; processing the sensed measurement parameters; and controlling a gas supply of at least one gas to the microfluidic structure based upon the processed measurement parameters.

While the invention has been illustrated and described in connection with currently preferred embodiments shown and described in detail, it is not intended to be limited to the details shown since various modifications and structural changes may be made without departing in any way from the spirit and scope of the present invention. The embodiments were chosen and described in order to explain the principles of the invention and practical application to thereby enable a person skilled in the art to best utilize the invention and various embodiments with various modifications as are suited to the particular use contemplated.

What is claimed is:

1. A system comprising:
a microfluidic lid assembly configured to create an air-tight seal above a sample container having reservoirs, the microfluidic lid assembly comprising:
guide elements;
a layer with apertures configured to align underneath the guide elements; and
a microfluidic structure with through-holes configured to align underneath the apertures of the layer, wherein the microfluidic structure comprises:
gas inlets configured to fluidly couple with one or more fluid sources; and
microfluidic channels configured to fluidly couple the gas inlets to the reservoirs of the sample container.

2. The system of claim 1, wherein the microfluidic structure is configured to individually seal each of the reservoirs of the sample container.

3. The system of claim 2, wherein each microfluidic channel is configured transport a controlled gas concentration to an individually sealed one of the plurality of reservoirs.

4. The system of claim 1, wherein a first subset of the microfluidic channels is configured to convey one or more of gaseous oxygen, nitrogen, or carbon dioxide to the reservoirs.

5. The system of claim 4, wherein a second subset of the microfluidic channels is configured to convey liquid reagents to the reservoirs.

6. The system of claim 1, wherein the microfluidic structure further comprises additional microfluidic channels configured to convey a gas away from the reservoirs.

7. The system of claim 1, wherein the guide elements and the layer form an integral unit.

8. The system of claim 1, wherein the guide elements are disposed on a guide structure that is coupled to the layer.

9. The system of claim 1, wherein the microfluidic lid assembly is configured to be adhered to the sample container with an adhesive.

10. The system of claim 1, wherein the apertures comprise slits in the layer.

11. The system of claim 1, wherein the layer comprises a resilient polymer material.

12. The system of claim 1, further comprising:
a sample container assembly, comprising:
the sample container comprising the reservoirs; and
the microfluidic structure, wherein a bottom surface of the microfluidic structure is adhered to a top surface of the sample container.

13. The system of claim 12, wherein a top surface of the microfluidic structure is adhered to a bottom surface of the layer.

14. The system of claim 12, further comprising:
a bioreactor system, comprising:
the sample container assembly;
a shaking table configured to shake the sample container assembly by moving the sample container assembly within a predetermined range of motion, wherein the predetermined range of motion is within an interior diameter of a top end of a guide element; and
an automated pipettor comprising one or more pipettors configured to insert one or more pipette tips into the sample container via the guide element while the sample container assembly is being shaken.

15. The system of claim 14, wherein the bioreactor system further comprises:
an upper chamber disposed above the shaking table; and
a cover inlay configured to direct tempered air in the upper chamber to uniformly temper each of the reservoirs.

16. The system of claim 15, wherein the cover inlay includes vent holes that align with the reservoirs, the vent holes configured to direct the tempered air.

17. The system of claim 14, wherein the bioreactor system further comprises:
a lower chamber disposed below the shaking table; and
one or more fans configured to circulate tempered air around the lower chamber.

18. The system of claim 14, wherein the bioreactor system further comprises:
an upper chamber disposed above the shaking table;
a lower chamber disposed below the shaking table;
one or more first temperature control modules configured to temper air of the upper chamber at a first target temperature; and
one or more second temperature control modules configured to temper air of the lower chamber at a second target temperature.

19. The system of claim 18, wherein the first temperature is set higher than the second temperature to prevent condensation in the bioreactor system.

20. The system of claim 14, further comprising:
an automatic cell culture system, comprising:
a titer module; and
the bioreactor system, wherein the bioreactor system includes cell health and cell media measurement capabilities integrated with the titer module.

21. The system of claim 12, further comprising:
a control system, comprising:
sensors configured to acquire measurement parameters associated with the sample container assembly;
a gas supply system configured to provide at least one gas to the microfluid structure; and
a controller configured to process the acquired measurement parameters and control the gas supply system based upon the processed measurement parameters.

22. A method comprising:
attaching a microfluidic structure to a top surface of a sample container;
attaching a resilient layer to a top surface of the microfluidic structure; and
attaching at least one guide element to a top surface of the resilient layer.

23. The method of claim 22, further comprising:
adhering the microfluidic structure to the top surface of the sample container.

24. The method of claim 22, further comprising:
shaking the sample container;
actuating a robot arm to guide a pipette tip to a narrowest region of the at least one guide element; and
guiding the pipette tip through the narrowest region of the at least one guide element into the sample container.

25. The method of claim 22, further comprising:
placing the sample container with the microfluidic structure attached to the top surface of the sample container within an anaerobic environment;
disposing a sample comprising anaerobic cells into one or more reservoirs of the sample container while the sample container is in the anaerobic environment;
creating an air-tight seal around the reservoirs of the sample container by placing a lid assembly over the reservoirs of the sample container; and
transporting the sealed sample container to a non-anaerobic environment for cell cultivation.

26. The method of claim 22, further comprising:
placing a microfluidic lid assembly above the sample container, the sample container including reservoirs, the microfluidic lid assembly including the microfluidic structure, the resilient layer, and the at least one guide element, the microfluidic lid assembly configured to provide a headspace above the reservoirs to allow gas exchange during cell cultivation, wherein the headspace above the reservoirs is 20 mL to 400 mL; and
causing a gas to flow into the headspace.

27. The method of claim 26, further comprising:
placing the sample container within an anaerobic environment;
disposing a sample comprising anaerobic cells into one or more reservoirs of the sample container while the sample container is in the anaerobic environment;
creating an air-tight seal around the reservoirs of the sample container by attaching the microfluidic lid assembly to the top surface of the sample container; and
transporting the sealed sample container to a non-anaerobic environment for cell cultivation.

28. The method of claim 22, further comprising:
sensing measurement parameters associated with a sample container assembly comprising the sample container and the microfluidic structure;
processing the sensed measurement parameters; and
controlling a gas supply of at least one gas to the microfluidic structure based upon the processed measurement parameters.
